(19) United States
(12) Patent Application Publication
(10) Pub. No.: US 2010/0209957 A1
(43) Pub. Date: Aug. 19, 2010

(54) Biosample storage devices and methods of use thereof

(75) Inventors: Michael Hogan, Tucson, AZ (US); Ferdinand Stupka, San Diego, CA (US); David Wong, Escondido, CA (US)

Correspondence Address: COOLEY LLP;ATTN: Patent Group, Suite 1100, 777 - 6th Street, NW, WASHINGTON, DC, 20001, US

(73) Assignee: GENVAULT CORPORATION, Carlsbad, CA (US)

(21) Appl. No.: 12/489,400

(22) Filed: Jun. 22, 2009

Related U.S. Application Data
(60) Provisional application No. 61/074,471, filed on Jun. 20, 2008.
(60) Provisional application No. 61/140,829, filed on Dec. 24, 2008.
(60) Provisional application No. 61/142,874, filed on Jan. 6, 2009.

(51) Int. Cl. C12Q 1/02; G01N 1/28; B01L 3/00; B01L 9/00; C12M 1/00; G01N 1/00

(52) U.S. Cl. 435/29; 422/102; 422/101; 422/58; 422/104; 422/61; 435/307.1; 436/174

(57) ABSTRACT

The present invention provides sample collection, shipping, and storage devices and methods of using the same. These devices and methods are useful, for example, for collecting, shipping, and storing biological samples, such as blood, serum, buccal samples, tissue homogenates, or cell lysates in a dry state. The devices and methods facilitate the rapid drying of biological samples collected on the devices, thereby improving the quality of the stored sample, particularly the protein and small molecule components of the stored sample. The present invention further provides methods of recovering biological samples from such devices.

The present invention claims priority from U.S. Provisional Application No. 61/074,471, filed on Jun. 20, 2008, U.S. Provisional Application No. 61/140,829, filed on Dec. 24, 2008, and U.S. Provisional Application No. 61/142,874, filed on Jan. 6, 2009, the contents of each of which is expressly incorporated herein by reference.

### FIELD OF THE INVENTION

This invention relates generally to devices for the collection, shipping, and storage of biological samples, such as blood, serum, milk, and tissue homogenates, and methods of using such devices to collect, ship, store, and retrieve biological samples.

### BACKGROUND OF THE INVENTION

In many applications, such as medical testing, pharmaceutical and medical research, law enforcement, and military identification, it is often desirable to have access to numerous biological samples. Conventional biorepositories or other sample storage facilities typically utilize liquid or low temperature cryogenic systems for sample storage. These liquid and cryogenic systems are expensive both to create and to maintain. Additionally, current technology generally presents system operators with complicated and labor intensive maintenance and administrative responsibilities.

Recently, biological research laboratory systems have been proposed which incorporate archiving and retrieval of blood samples in dry or desiccated form. Present systems are generally based upon modifications or variations of known techniques for storing DNA or other organic samples on a suitable substrate such as filter paper. Some systems require, or substantially benefit from, soaking the substrate or paper with chemical denaturants and detergents prior to use.

The process of drying biological samples presents complications, however, because various biological molecules present in the samples can become denatured or damaged during the process. For example, the drying of biological samples is typically performed at room temperature (or even higher temperatures) and enzymes that can damage biological molecules, such as proteases and nucleases, are active at those temperatures. In addition, the temperatures used for drying allow for contamination by microorganisms, such as bacteria or yeast, that can further damage the biological samples.

Accordingly, there remains a need in the art for devices that provide for the collection and storage of biological samples in the dry state.

### SUMMARY OF THE INVENTION

The present invention is based, in part, on the development of sample carriers that can be used to collect biological samples in medical, veterinary, and other field settings, and to ship and store such samples. The present invention is also based, in part, on the discovery that biological samples, particularly those containing proteins and small molecule components, are better preserved in a dry state when they are dried rapidly. Accordingly, in one aspect, sample carriers suitable for collection, shipping, and/or storage of biological samples are provided. In certain embodiments, the sample carriers comprise an opening configured to hold a sample node. The opening can, for example, have a side surface that contacts, and thereby holds (e.g., by pressure or adhesive contacts), a sample node. Alternatively, the opening can provide a post (or outwardly pointing protrusion) that contacts, and thereby holds, a sample node. In certain embodiments, the opening provides one or more ventilation spaces that facilitate evaporation and/or air flow at the surface of a sample node. Additionally, or in the alternative, the opening can provide one or more sub-opening spaces to a surface of a sample node. The sub-opening spaces can facilitate evaporation and/or air flow at the surface of the sample node. Additionally, or in the alternative, the opening can comprise a plurality of protrusions (e.g., inwardly pointing protrusions) designed to contact, and thereby hold (e.g., by pressure or adhesive contacts), a sample node. The space between protrusions can provide a ventilation space that facilitates evaporation and/or air flow at the surface of the sample node.

In certain embodiments, sample carriers of the invention comprise a sealing mechanism, wherein the sealing mechanism is positioned so as to interface with a corresponding receptacle, thereby creating a sealed chamber around the opening for the sample node. In certain embodiments, the sealing mechanism comprises a screw mechanism, such as threading, or a friction-based locking mechanism, such as a lip or hook capable of engaging a groove or notch in a corresponding receptacle. In certain embodiments, sample carriers that have been sealed can be externally sterilized.

In certain embodiments, sample carriers of the invention can further comprise a plurality of openings, each configured to hold a sample node. The openings can be configured in an array, such as a rectilinear array. In addition, sample carriers can comprise an identifying indicia, such as a barcode or radio frequency tag.

In another aspect, sample carriers comprising an opening and a sample node are provided. The opening can be any type of opening described herein. In certain embodiments, the sample node is held by the opening. In certain embodiments, the sample node is held by the opening such that the surface area of the sample node contacted by the opening is minimized. For example, in certain embodiments, when held by an opening of a sample carrier, at least 60%, 65%, 70%, 75%, 80%, 85%, 90%, 95%, or more of the surface area of a sample node can be exposed to air. In certain embodiments, the opening has greater depth than the height of the sample node. In certain related embodiments, when the sample node is held by the opening, the top surface of the sample node is flush with or recessed relative to the top surface of the sample carrier. In other nonexclusive embodiments, when the opening has greater depth than the height of the sample node a portion of the opening located beneath the bottom surface of the sample node forms a reservoir (e.g., when the opening has a concave topology).

In certain embodiments, the sample node comprises a substrate suitable for dry state storage of biological samples. For example, in certain embodiments, a sample node comprises a macroporous medium. The macroporous medium can be elastomeric and/or have an open cell foam structure. Alternatively, the macroporous medium can comprise cellulose (e.g., filter paper) and/or have an open pore structure. In certain embodiments, the sample node comprises a stabilizer, such as a filler, a wetting agent, a reactive oxygen scavenger (ROS), a detergent, a metal chelating agent, and/or a buffer. In certain embodiments, the sample node comprises an identifying indicia, such as a biological coding composition.

In certain embodiments, sample carriers of the invention further comprise a plurality of openings and a plurality of sample nodes, wherein each sample node is held by a corresponding (e.g., single) opening.

In another aspect, sample carriers comprising an opening, a sample node, and a biological sample are provided. The opening can be any type of opening described herein. Similarly, the sample node can be any type of sample node descried herein. In certain embodiments, the biological sample is carried by (e.g., absorbed to) the sample node. Exemplary biological samples include blood, serum, plasma, buccal samples, sputum samples, nasal swab samples, milk, homogenized animal or plant tissues, and cell lysates. The biological samples can be from any biological organism, including humans, farm animals, zoo animals, laboratory animals, wild animals, microorganisms, viruses, etc.

In certain embodiments, sample carriers of the invention further comprise a plurality of openings, a plurality of sample nodes, and one or more biological samples, wherein each sample node is held by a corresponding (e.g., single) opening, and wherein each biological sample is carried by a corresponding (e.g., single) sample node.

In another aspect, storage systems are provided. In certain embodiments, a storage system comprises a sample carrier and a receptacle. The sample carrier can be any type of sample carrier described herein. In certain embodiments, the receptacle is suitable for storing and/or sealing one or more sample carriers. For example, one or more sample carriers can be placed into a receptacle and the receptacle can be stored in an archive. Alternatively, or in addition, a receptacle can comprise a sealing mechanism configured to engage a sample carrier, thereby creating a sealed chamber around the opening for the sample node and any sample node held thereby. The sealing mechanism can be, for example, a screw mechanism, such as threading (e.g., capable of interlocking with threading on the sample carrier), or a friction-based locking mechanism, such as a groove or notch designed to receive a lip or hook located on the sample carrier. In certain embodiments, a receptacle comprises a drying agent, such as a desiccant. In certain embodiments, a receptacle comprises an identifying indicia, such as a barcode.

In certain embodiments, storage systems of the invention comprise a plurality of sample carriers and one or more receptacles, wherein each receptacle is suitable for storing and/or sealing one or more sample carriers.

In another aspect, methods of collecting biological samples are provided. In certain embodiments, the methods comprise applying, directly or indirectly, a biological sample to a sample node of a sample carrier. The sample node and sample carrier can be any sample node and sample carrier described herein. The biological sample can be, for example, blood, serum, plasma, a buccal sample, a sputum sample, a nasal swab sample, milk, homogenized animal or plant tissues, or a cell lysate. The biological sample can be fresh, such as a blood sample obtained using a finger stick or a heel stick, or milk taken from a nipple or udder. Alternatively, the biological sample can be one that was collected previously, e.g., hours, days, or months ago.

In certain embodiments, the methods of collecting biological samples comprise drying a sample node to which a biological sample has been applied. The sample node can be held by an opening in a sample carrier during the drying process. In certain embodiments, the drying of the sample node is facilitated. Facilitated drying can be accomplished, for example, by placing the sample node (e.g., held by a sample carrier) into a low humidity chamber, providing air circulation around the sample node, and/or sealing the sample carrier with a receptacle that comprises a desiccant (e.g., held in close proximity to the sample node).

In certain embodiments, the methods of collecting biological samples comprise recording an identifying indicia associated with the biological sample. Identifying indicia associated with a biological sample can include, for example, identifying indicia from a sample carrier (e.g., a barcode or radio frequency tag) and/or a sample node that the biological sample is stored upon (e.g., a biological coding composition). Identifying indicia associated with a biological sample can be recorded on paper medium or electronic medium, such as a computer. The record thus created can be stored in a data repository, such as a file, or in a computer database.

In certain embodiments, the methods of collecting biological samples further comprise externally sterilizing a sample carrier after the sample has been collected. In certain embodiments, the sample carrier is sealed, for example, by interfacing with a corresponding receptacle, prior to the external sterilization. In certain embodiments, the sterilization is achieved by rinsing or wiping down the sample carrier (and receptacle, as appropriate) with a chemical suitable for such purposes (e.g., rubbing alcohol or other organic sterilizer). In other embodiments, the sterilization is achieved using radiation or other non-chemical means.

In certain embodiments, the methods of collecting biological samples further comprise shipping the sample carrier after the biological sample has been collected. In certain embodiments, such shipping is facilitated by use of a receptacle. The receptacle can be any receptacle described herein. For example, the sample carrier can be sealed using a receptacle suitable for interfacing and sealing the sample carrier. The resulting sample carrier-receptacle combination (e.g., storage system) can be shipped from the place where the sample is collected to the place where the sample will be stored and/or recovered. In certain embodiments, the shipping comprises tracking the location of the sample carrier at one or more intermediate locations in the shipping route. In certain embodiments, the sample carrier is externally sterilized prior to being shipped.

In another aspect, methods of storing a biological sample are provided. In certain embodiments, the methods comprise storing a sample carrier comprising a biological sample in an archive. The sample carrier and biological sample can be any sample carrier and biological sample described herein. In certain embodiments, the sample carrier is placed into a receptacle (e.g., a receptacle described herein), prior to being stored. The receptacle holding the sample carrier can then be placed into an archive. In certain embodiments, the sample carrier is sealed (e.g., by interfacing with a corresponding receptacle) and/or sterilized prior to being stored.

In certain embodiments, the methods of storing a biological sample comprise recording an identifying indicia associated with the biological sample. Identifying indicia associated with a biological sample can include, for example, identifying indicia from a receptacle (e.g., a barcode), a sample carrier (e.g., a barcode), and/or a sample node that the biological sample is stored in or upon (e.g., a coding composition). Identifying indicia associated with a biological sample can be recorded on paper medium or electronic medium. The record thus created can be stored in a data repository, such as a file or a database.

In certain embodiments, the methods of shipping and/or storing a biological sample further comprise recovering the biological sample after it has been shipped and/or stored.

In another aspect, methods of recovering a biological sample are provided. In certain embodiments, the methods of recovering a biological sample comprise rehydrating a sample node carrying the sample. The biological sample and sample node can be any biological sample and sample node described herein. In certain embodiments, rehydrating a sample node comprises adding a fluid, such as water or an appropriate buffer, to the sample node. In other embodiments, rehydrating a sample node comprises adding a fluid, such as water or an appropriate buffer, to the opening of a sample carrier, wherein the opening is holding the sample node. In certain embodiments, the rehydrating fluid is a wash buffer. In other embodiments, the rehydrating fluid is an elution buffer.

In certain embodiments, rehydrating fluid (e.g., wash buffer or elution buffer containing biological sample) is separated from the sample node following the rehydration step. For example, the sample node can be compressed and/or centrifuged to separate away the rehydrating fluid. In certain embodiments, the sample carrier comprises a reservoir located beneath the sample node, wherein the reservoir facilitates separation of the rehydrating fluid from the sample node.

In certain embodiments, rehydrating fluid obtained from a sample node typically contains molecules of interest originating from the biological sample, such as DNA, RNA, protein, lipids, hormones, small molecule analytes, drugs, and other biological molecules.

In certain embodiments, the methods of recovering a biological sample comprise removing a sample carrier comprising the sample node from a receptacle and/or breaking a seal formed between the sample carrier and a corresponding receptacle. In certain embodiments, the methods of recovering a biological sample comprise removing a sample node carrying the biological sample from a sample carrier that holds the sample node. The sample node and sample carrier can be any sample node and sample carrier described herein. In certain embodiments, removing a sample node from a sample carrier comprises pushing the sample node out of an opening in the sample carrier. In other embodiments, removing a sample node from a sample carrier comprises pulling the sample node from an opening in the sample carrier. The step of removing the sample node from the sample carrier can occur before or after the sample node has been rehydrated.

In yet another aspect, kits for collecting, shipping, and/or storing biological samples are provided. In certain embodiments, the kits comprise a sample carrier of the invention. In other embodiments, the kits comprise a storage system of the invention.

### BRIEF DESCRIPTION OF THE DRAWINGS

FIG. 1 is a diagram of one embodiment of a sample carrier of the invention. The sample carrier has six openings, each of which is circular and has six sub-opening spaces.

FIG. 2 is a diagram of the sample carrier of FIG. 1 comprising tube-like, cylindrical sample nodes, wherein each opening of the sample carrier is holding a sample node.

FIG. 3 is a diagram of one embodiment of a receptacle of the invention. The receptacle can receive up to six sample carriers of the type shown in FIG. 1.

FIG. 4 is a diagram of one embodiment of a storage system of the invention. The storage system comprises the receptacle of FIG. 3 and six sample carriers of the type shown in FIG. 1.

FIG. 5 is a diagram of another embodiment of a storage system of the invention. In this embodiment, the sample carrier comprises a cup-like topology and a sealing mechanism featuring threading designed to interface with a threading on a corresponding receptacle. The sample carrier is designed to hold a single sample node via three ridge-like protrusions, with ventilation spaces created in the space bounded by the protrusions, the side surface of the opening and the side surface of the sample node. The corresponding receptacle is designed to hold a desiccant to facilitate drying of the sample node after the sample carrier is sealed.

FIG. 6 is a diagram of one embodiment of a tray having a standard SBC format and capable of holding 12 sample carriers of the type described in FIG. 5.

FIG. 7 is a diagram of yet another storage system of the invention. This storage system is similar to the one of FIG. 5, but enlarged to allow for collection of larger samples.

FIG. 8 is a gel showing the results of DNA recovered from whole blood applied to sample nodes comprising an elastomer substrate. The whole blood was allowed to dry on the elastomer, then stored at room temperature or 56° C. for up to 34 days.

FIG. 9 is a gel showing the results of 10 kb mitochondrial DNA PCR performed on the DNA samples of FIG. 8.

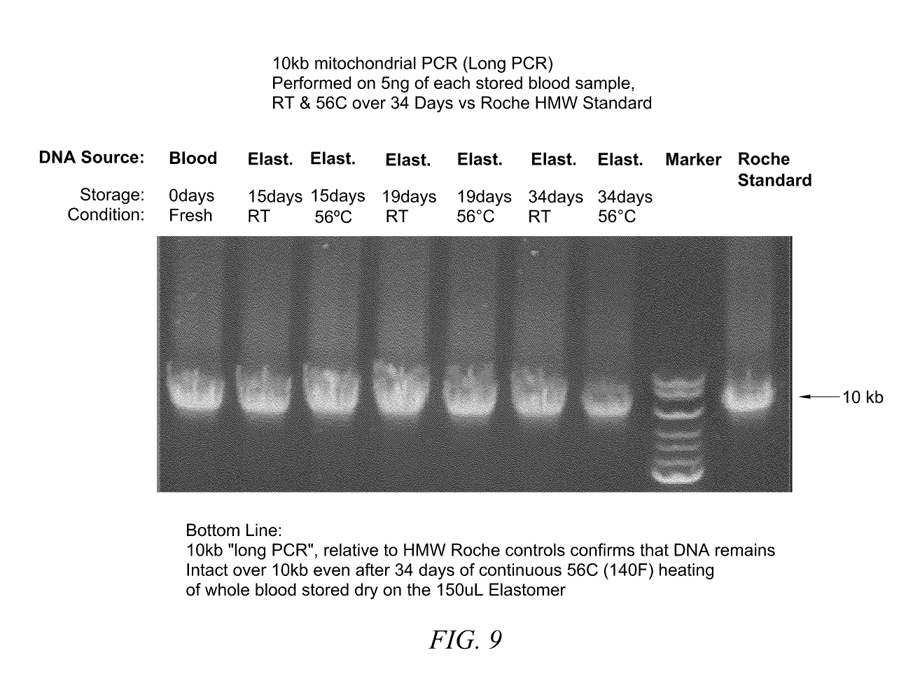

FIG. 10 is a graph showing the results of protein recovered from serum, plasma, and whole blood dried upon samples nodes comprising an elastomer substrate and stored at 25° C. for 28 days.

### DETAILED DESCRIPTION OF THE INVENTION

The present invention provides sample collection, shipping, and storage devices, and methods of using the same. These devices and methods are useful, for example, for collecting, shipping, and/or storing biological samples, such as blood, serum, buccal samples, milk, tissue homogenates, or cell lysates in a dry state. The devices and methods facilitate the rapid drying of biological samples applied to sample nodes in the devices, thereby improving the quality of the stored biological samples, particularly the protein and small molecule components of such samples. The present invention further provides methods of recovering samples, such as biological samples, from such devices.

Accordingly, in one aspect, the invention provides a sample carrier comprising an opening. The term “opening,” as used herein, refers to a partially enclosed space defined by a surface of an object, such as a surface of a sample carrier. In certain embodiments, an opening is a space that extends through an object, like a tunnel. In other embodiments, an opening is a cavity in an object. In certain embodiments, the cavity is at least partially defined by a surface (e.g., a surface defining the bottom of the cavity) that is porous. For example, in certain embodiments, the cavity is at least partially defined by a porous surface, wherein the porous surface comprises a plurality of pores having a cross-sectional size smaller (e.g., by a factor of 1/10, 1/20, 1/25, etc.) than the cross-sectional size of the cavity. In other embodiments, the cavity is at least partially defined by a surface (e.g., a surface defining the bottom of the cavity) that comprises or consists of a gas permeable membrane.

In certain embodiments, the sample carrier comprises an opening, wherein the opening is configured to hold a sample node. The term “configured,” when used herein to refer to an opening, means that the opening is structured or designed in an operative way to hold a sample node, e.g., via the shape of the opening, a mechanical design of the opening, adhesive contacts located at one or more places in the opening, a device (e.g., a post) included in the opening, or a combination thereof. In certain embodiments, the opening is configured to hold a single sample node. In other embodiments, the opening is configured to hold a plurality of sample nodes.

In certain embodiments, the configuration of the opening comprises a circle. For example, the opening can have a circular shape in cross-section. In other embodiments, the configuration of the opening comprises a polyhedral shape (e.g., a regular or irregular polyhedral shape), such as a triangle, square, rectangle, pentagon, hexagon, etc. In certain embodiments, the width of the opening remains roughly constant throughout the depth of the passage or cavity that defines the opening. Thus, for example, the opening can have a columnar shape that comprises either a circular or polyhedral shape in cross-section. In other embodiments, the width of the opening varies with the depth of the passage or cavity that defines the opening.

The term “to hold,” when used herein to refer to a function of an opening, means that the object being held is securely positioned in a particular location. For example, a sample node that is held by the opening of a sample carrier will typically not be dislodged during routine handling of the sample carrier, e.g., if the sample carrier is turned over or jolted during shipping and/or handling. In certain embodiments, an opening of a sample carrier holds a sample node by means of pressure contact(s). In such embodiments, the force required to dislodge the sample node from the opening is the force required to overcome the friction that resists sliding of the sample node past such contact(s). In other embodiments, an opening of a sample carrier holds a sample node by means of adhesive contact(s), e.g., localized contact(s) mediated by a glue or other cement. In such embodiments, the force required to dislodge the sample node from the opening is the force required to break the chemical structure of the adhesive contact(s) that resists sliding of the sample node past such contact(s). Suitable glues or cements include, but are not limited to, epoxy, silicone and protein based glues. In still other embodiments, an opening of a sample carrier holds a sample node by means of pressure and adhesive contact(s). The present disclosure is not intended to be limited to any particular contact design, type of contact, glue or cement. Persons skilled in that art will recognize that many different types of contacts can be used that enable a sample carrier to hold a sample node, depending upon the intended use of the sample carrier.

In certain embodiments, an opening in a sample carrier comprises a side surface that contacts the sample node, wherein the sample node is held by the side surface contact(s). The term “side surface,” as used herein in reference to an opening, is a surface of the sample carrier that defines the opening. For example, if an opening in a sample carrier has a cylindrical shape that passes through the sample carrier, a side surface of the opening is the corresponding cylindrically shaped surface on the sample carrier that defines the opening. In certain embodiments, a contact between a side surface of an opening and a sample node comprises an interface having an area of about 2 mm² to about 15 mm², about 3 mm² to about 10 mm², about 4 mm² to about 8 mm², or about 5 mm². In other embodiments, a contact between a side surface of an opening and a sample node comprises an interface having an area of about 4 mm² to about 30 mm², about 6 mm² to about 20 mm², about 8 mm² to about 16 mm², or about 10 mm².

In certain embodiments, an opening has a cross-sectional area of about 10 mm² to about 100 mm², about 15 mm² to about 95 mm², about 20 mm² to about 90 mm², about 25 mm² to about 85 mm², about 30 mm² to about 80 mm², or about 35 mm² to about 75 mm². In other embodiments, an opening has a cross-sectional area of about 15 mm² to about 150 mm², about 30 mm² to about 140 mm², about 45 mm² to about 130 mm², about 60 mm² to about 120 mm², about 75 mm² to about 110 mm², or about 90 mm² to about 100 mm².

In certain embodiments, an opening in a sample carrier is configured to hold a sample node while providing a ventilation space to the sample node. A “ventilation space,” as used here, is a space within an opening that is unoccupied by a sample node being held by the opening. For example, if a sample carrier has an opening which is square in cross-section and a cylindrical sample node is being held by the square opening such that the surface of the sample carrier defining the opening contacts the sample node in four discrete locations (i.e., one contact on each side of the square opening), four ventilation spaces will be formed. In cross-section, the four ventilation spaces formed comprise the four discrete areas formed between a circle and a square when a circle is inscribed within the square. Similarly, if a sample carrier has a rectangular opening and a cylindrical sample node is being held by the opening such that the surface of the sample carrier defining the opening contacts the sample node in two discrete locations (i.e., one contact on each of two opposite sides of the rectangular opening), there will be two ventilation spaces formed.

In certain embodiments, a ventilation space has a cross-sectional area of about 1 mm², 1.5 mm², 2 mm², 2.5 mm², 3 mm², 3.5 mm², 4 mm², 4.5 mm², 5 mm², 6 mm², 7 mm², 8 mm², 9 mm², 10 mm², 12 mm², 14 mm², 16 mm², 18 mm², 20 mm², or more. In certain embodiments, a ventilation space has a volume of about 5 mm³, 10 mm³, 15 mm³, 20 mm³, 25 mm³, 30 mm³, 35 mm³, 40 mm³, 45 mm², 50 mm³, 55 mm³, 60 mm³, 65 mm³, 70 mm³, 75 mm³, 80 mm³, 85 mm³, 90 mm³, 95 mm³, 100 mm³, or more.

In certain embodiments, a ventilation space facilitates (i.e., increases) fluid evaporation at the surface of a sample node. In certain embodiments, a ventilation space increases the rate of fluid evaporation at the surface of a sample node by at least 10%, 20%, 30%, 40%, 50%, 60%, 70%, 80%, 90%, 100%, 110%, 120%, 130%, 140%, 150%, 160%, or more. In other embodiments, a sub-opening space facilitates (i.e., increases) air flow at the surface of a sample node.

In certain embodiments, an opening in a sample carrier comprises two or more (e.g., 2, 3, 4, 5, 6, or more) ventilation spaces when a sample node is held by the opening.

In certain embodiments, the sample carrier comprises an opening, wherein the opening is configured to hold a plurality of sample nodes. For example, a sample carrier can have a rectangular opening that is able to hold two or more sample nodes. Preferably, in such embodiments, adjacent sample nodes do not contact one another.

In certain embodiments, the sample carrier comprises an opening, wherein the opening is configured to hold a sample node while providing a sub-opening space to a surface of the sample node. The term “sub-opening space,” as used herein, refers to an additional or expanded space that enlarges the space otherwise provided by an opening, such as a secondary opening from the surface of an opening. For example, a “sub-opening space” can be a space that extends out from an opening such that the gap between the surface of the sub-opening space and the surface of a sample node held by the opening is greater (i.e., greater on average) than the gap between the surface of the opening and the surface of the sample node if the sub-opening space was not present. Thus, in certain embodiments, a sub-opening space is a secondary opening from the surface of an opening's primary shape. In certain embodiments, a sub-opening space is a secondary opening which expands the space provided by an opening's primary or designated shape. For example, if an opening's primary shape is cylindrical, a sub-opening space could be a secondary opening from the cylindrical opening which expands the space otherwise provided by the opening.

Typically, a sub-opening space decreases the amount of sample node surface area contacted by an opening that is holding the sample node and/or increases the volume of air located adjacent to the surface of a sample node (e.g., increases the volume of air located within 1 mm, 2 mm, 3 mm, 4 mm, 5 mm, etc. of the surface of a sample node) that is being held by the opening. Persons skilled in the art will understand that the precise shape of an opening and a sub-opening space are not critical provided that the opening is capable of holding a sample node and the sub-opening space decreases the amount of sample node surface area contacted by an opening that is holding the sample node, e.g., by 1%, 2%, 3%, 4%, 5%, 10%, 15%, 20%, 25%, 30%, 35%, 40%, 45%, 50%, 60%, 70%, 80%, 90%, or more, and/or increases the volume of air located adjacent to the surface of the sample node, e.g., by 1%, 2%, 3%, 4%, 5%, 10%, 15%, 20%, 25%, 30%, 35%, 40%, 45%, 50%, 60%, 70%, 80%, 90%, or more. The increase in air volume can be measured by comparing the volume of air located within 1.0 mm, 1.5 mm, 2.0 mm, 2.5 mm, 3.0 mm, 3.5 mm, 4.0 mm, 4.5 mm, or 5.0 mm of the surface of a sample node held by a first opening comprising a sub-opening space and a second opening having the same primary shape as the first opening but lacking the sub-opening space.

In certain embodiments, a sub-opening space has a cross-sectional area of at least 4 mm², 5 mm², 6 mm², 7 mm², 8 mm², 9 mm², 10 mm², 15 mm², 20 mm², or more. In certain embodiments, a sub-opening space has a volume of at least 25 mm³, 30 mm³, 35 mm³, 40 mm³, 50 mm³, 55 mm³, 60 mm³, 65 mm³, 70 mm³, 75 mm³, 80 mm³, 85 mm³, 90 mm³, 95 mm³, 100 mm³, or more. In certain embodiments, a sub-opening space has an increasingly larger width outwards of the center of the opening. In certain embodiments, a sub-opening space passes through a sample carrier, like a tunnel. In other embodiments, a sub-opening space is a cavity in a sample carrier.

In certain embodiments, a sub-opening space facilitates (i.e., increases) fluid evaporation at the surface of a sample node. In certain embodiments, a sub-opening space increases the rate of fluid evaporation at the surface of a sample node by at least 10%, 20%, 30%, 40%, 50%, 60%, 70%, 80%, 90%, 100%, 110%, 120%, 130%, 140%, 150%, 160%, or more. In other embodiments, a sub-opening space facilitates (i.e., increases) air flow at the surface of a sample node.

In certain embodiments, an opening in a sample carrier comprises two or more sub-opening spaces. In certain embodiments, an opening comprises an open circle, e.g., a circular shape in cross-section that opens to at least 1, 2, 3, 4, 5, 6, or more sub-opening spaces. In certain embodiments, an opening comprises an open polyhedral, e.g., a regular or irregular polyhedral shape, such as a triangle, square, rectangle, pentagon, hexagon, etc., in cross-section that opens to at least 1, 2, 3, 4, 5, 6, or more sub-opening spaces. In certain embodiments, an opening in a sample carrier that comprises two or more sub-opening spaces increases the rate of evaporation at the surface of a sample node by 25%, 50%, 75%, 100%, 150%, 200%, 250%, 300%, 350%, 400%, 450%, 500%, 550%, 600%, 650%, 700%, 750%, 800%, 850%, 900%, 950%, 1000%, or more.

In certain embodiments, the sample carrier comprises an opening, wherein the opening comprises one or more protrusions. The term “protrusion,” as used herein, refers to a portion of the sample carrier that extends into the opening, starting from a surface of the sample carrier that defines the opening. In certain embodiments, the protrusion is inwardly pointing, e.g., the protrusion extends from a side surface of the opening toward the center of the opening or toward a central axis of the opening. In certain embodiments, the protrusion is outwardly pointing, e.g., the protrusion extends upward from a base surface of the opening (e.g., a base surface of an opening shaped like a cavity) to point outwards. In certain embodiments, a protrusion (e.g., an outwardly pointing protrusion) comprises solid or a non-solid structure. For example, a protrusion can have a circular or polyhedral shape in cross-section or, in the alternative, can have vanes or grooves that result in an irregular (e.g., asterisk-like) shape in cross-section.

In certain embodiments, the protrusion is configured to contact a sample node being held by the opening. In certain embodiments, the contact is a pressure contact, an adhesive contact, a hook, a lip, or a combination thereof. In certain embodiments, the contact between the protrusion and sample node assists with the holding of the sample node in the opening of the sample carrier. For example, in certain embodiments, a plurality of protrusions (e.g., inwardly-pointing protrusions) contact a sample node, thereby holding the sample node within the opening. In other embodiments, a single protrusion (e.g., outwardly-pointing protrusion) contacts the sample node (e.g., via a shaft-like opening in the sample node that permits insertion of the protrusion), thereby holding the sample node within the opening. In still other embodiments, a plurality of protrusions comprising both inwardly- and outwardly-pointing protrusions contact a sample node, thereby holding the sample node in place.

In certain embodiments, the contact between the protrusion and sample node comprises an interface having an area of about 1 mm² to about 3 mm², about 2 mm² to about 4 mm², about 3 mm² to about 6 mm², about 4 mm² to about 8 mm², about 5 mm² to about 10 mm², about 6 mm² to about 12 mm², about 7 mm² to about 14 mm², about 8 mm² to about 16 mm², about 9 mm² to about 18 mm², about 10 mm² to about 20 mm², about 11 mm² to about 22 mm², about 12 mm² to about 24 mm², about 13 mm² to about 26 mm², or about 14 mm² to about 28 mm².

In certain embodiments, a protrusion (e.g., an inwardly-pointing protrusion) extends about 0.5 mm to about 4.0 mm, about 0.75 mm to about 3.5 mm, about 1.0 mm to about 3.0 mm, about 1.2 mm to about 2.8 mm, about 1.4 mm to about 2.6 mm, about 1.6 mm to about 2.4 mm, about 1.8 mm to about 2.2 mm, or about 2.0 mm into the opening. In other embodiments, a protrusion (e.g., an outwardly-pointing protrusion) extends about 4.0 mm to about 16 mm, about 5.0 mm to about 15 mm, about 6.0 mm to about 14 mm, about 7.0 mm to about 13 mm, about 8.0 mm to about 12 mm, about 9.0 mm to about 11 mm, or about 10 mm into the opening.

In certain embodiments, an opening comprises a plurality of protrusions (e.g., 2, 3, 4, 5, 6, or more protrusions), e.g., defining or outlining one or more sub-opening spaces. For example, in certain embodiments, the space between adjacent protrusions (e.g., inwardly pointing protrusions) has the properties of a ventilation space or a sub-opening space of the present invention, as discussed supra. In certain embodiments, an opening comprises at least one outwardly-pointing protrusion that contacts and thereby holds the sample node, wherein a ventilation space is provided between a surface of the sample node and a side surface of the opening. For example, an opening and a sample node can each have a cylindrical shape, wherein the diameter of the opening is larger than the diameter of the cylinder, such that when the sample node is held by an outwardly-pointing protrusion originating from a bottom surface of the opening, there is no contact between the side surface of the opening and the side surface of the sample node. In such an instance, the space between the side surface of the opening and the side surface of the sample node constitutes a ventilation space.

In certain embodiments, an opening in a sample carrier is configured to hold a sample node while providing a space above and/or below the sample node. For example, in certain embodiments, the opening is configured to hold a sample node such that a top surface of the sample node is recessed relative to a top surface of the sample carrier. The opening can be configured so that the top surface of the sample node is recessed, for example, by 0.5 mm, 0.75 mm, 1.0 mm, 1.25 mm, 1.5 mm, or more.

In certain embodiments, the opening (e.g., a cavity) is configured to hold a sample node such that there is a reservoir defined by a portion of the opening located beneath the space designated for the sample node. In certain embodiments, such a reservoir has a volume at least as large (e.g., 100%, 105%, 110%, 115%, 120%, 125%, 130%, 135%, 140%, 145%, 150%, or more) as the sample node that the opening is designed to hold. In certain embodiments, such a reservoir has a volume of at least 160 mm³, 200 mm³, 250 mm³, 300 mm³, 350 mm³, 400 mm³, 450 mm³, 500 mm³, 600 mm³, 700 mm³, 800 mm³, 900 mm³, 1000 mm³, 1100 mm³, 1200 mm³, or more. In certain embodiments, such a reservoir can be separated from the sample node by means of an intervening layer, such as a porous layer that supports the sample node while allowing fluids (e.g., rehydrating fluid comprising recovered sample) to pass through into the reservoir. In certain related embodiments, the porous layer has sufficient mechanical strength and integrity to support the sample node during a centrifugation step used to separate fluid (e.g., rehydrating fluid comprising sample) from the sample node. In certain embodiments, the porous layer has a pore size of at least 1, 5, 10, 20, 30, 40, 50, or more microns.

In certain embodiments, the sample node is held by an opening (e.g., a cavity) in the sample carrier such that a top surface of the sample node is recessed relative to a top surface of the sample carrier and such that there is a reservoir defined by a portion of the opening located beneath the sample node.

In certain embodiments, a sample carrier comprises a sealing mechanism. The sealing mechanism can be, for example, a structure that interfaces with a corresponding structure in a second object (e.g., a receptacle), thereby creating an enclosed space surrounding an opening in the sample carrier and a sample node held by said opening. In certain embodiments, the sealing mechanism forms an air-tight seal and/or a fluid-impermeable seal. The sealing mechanism can be any mechanism suitable for forming the desired type of seal. For example, the sealing mechanism can comprise a screw mechanism, such as threading designed to screw into or onto complementary threading on a corresponding receptacle. Alternatively, or in addition, the sealing mechanism can comprise a friction-based locking mechanism, such as a lip or hook designed to fit into a complementary groove or notch in a corresponding receptacle. Conversely, the sealing mechanism can comprise a groove or notch designed to accept a complementary lip or hook on a corresponding receptacle. In certain embodiments, the sealing mechanism further comprises a gasket. The gasket, for example, can be made from rubber, silicone, neoprene, nitrile rubber, fiberglass, a plastic polymer, paper, etc. Persons skilled in the art will understand that the sealing mechanism can be designed in many different ways depending upon the intended purpose, structure, and overall dimensions of the sample carrier.

In certain embodiments, a sample carrier comprises a plurality of openings of the present invention. In certain embodiments, the plurality of openings forms an array, e.g., a m by n array, wherein m=1, 2, 3, 4, 5, 6, 7, 8, 9, 10, 11, 12, 13, 14, 15, 16, 17, 18, 19, 20, 21, 22, 23, 24, or more, and n=1, 2, 3, 4, 5, 6, 7, 8, 9, 10, 11, 12, 13, 14, 15, 16, or more. As will be understood by one skilled in the art, the dimensions of the array can be selected in accordance with the intended use of the sample carrier.

In certain embodiments, a sample carrier is planar. As used herein, the term “planar” means that the sample carrier has a uniform thickness (i.e., a thickness that varies within 10% of a set value) and is neither substantially convex nor substantially concave. In certain embodiments, a sample carrier has a uniform thickness greater than about 5 mm. In certain embodiments, a sample carrier has a uniform thickness of about 5 mm to about 15 mm, about 6 mm to about 12 mm, or about 7 mm to about 9 mm. As will be understood by one skilled in the art, the thickness of the sample carrier can be selected in accordance with its intended use.

In certain embodiments, a sample carrier has a unit width of about 7 mm to about 16 mm, about 9 mm to about 14 mm, or about 11 mm to about 12 mm. In other embodiments, a sample carrier has a unit width of about 12 mm to about 21 mm, about 14 mm to about 19 mm, or about 15 mm to about 17 mm. A “unit width,” as used herein, refers to the width of a sample carrier that has a single opening. Thus, e.g., the width of a sample carrier having an array of openings can be calculated by multiplying these ranges by the number of openings in a row.

In certain embodiments, a sample carrier has a unit height of about 7 mm to about 16 mm, about 9 mm to about 14 mm, or about 11 mm to about 12 mm. In other embodiments, a sample carrier has a unit height of about 12 mm to about 21 mm, about 14 mm to about 19 mm, or about 15 mm to about 17 mm. A “unit height,” as used herein, refers to the height of a sample carrier that has a single opening. Thus, e.g., the height of a sample carrier having an array of openings can be calculated by multiplying these ranges by the number of openings in a column.

In certain embodiments, a sample carrier has a width or a height that is longer than the unit width or height, respectively, or a multiple thereof. For example, in certain embodiments, a sample carrier comprises additional area that is free of openings. Thus, openings in the sample carrier can have an asymmetric arrangement, wherein the asymmetric arrangement provides for an additional area. Such additional area can be located, for example, at one edge of the sample carrier. Such additional area can be used, for example, to grip the sample carrier and/or to provide a location for an identifying indicia. As will be understood by one skilled in the art, the height and width of the sample carrier can be selected in accordance with its intended use.

In certain embodiments, a sample carrier of the invention comprises a cup-like topology, wherein the interior space of the cup corresponds to an opening (e.g., an opening configured to hold a sample node). In certain embodiments, the sample carrier comprises a cup-like topology, wherein the opening is configured to hold a sample node, wherein a reservoir is formed by a portion of the opening located beneath the sample node, and wherein the sample carrier further comprises a sealing mechanism.

In certain embodiments, a sample carrier comprises an identifying indicia. An “identifying indicia,” as used herein in the context of a sample carrier, is anything that helps to identify the sample carrier and/or any sample nodes held by the sample carrier. Examples of such identifying indicia include, but are not limited to, hand-written information (e.g., patient identification information), a label (e.g., a typed or computer-generated label), a bar code (e.g., a one- or two-dimensional bar code that can be read by an optical scanner), a radio-frequency (RF) tag, a transceiver (e.g., a transceiver that is responsive to a query signal and emits an identification signal in response to the query signal), and/or a coding composition. Suitable coding compositions are disclosed, for example, in U.S. Patent Application No. 2005/0026181. The present disclosure is not intended to be limited to any particular identifying indicia. Persons skilled in the art will recognize that many different identifying indicia can be used in conjunction with sample carriers of the invention, depending upon their intended use.

Sample carriers can be made from any material or combination of materials having sufficient mechanical integrity to allow for handling by hand and/or machine (e.g., a machine that archives samples and/or retrieves samples from an archive). Materials that can be used to make sample carriers include, but are not limited to plastics, ceramics & metals which can be molded or milled to provide openings of the present invention, including, e.g., openings comprising sub-opening spaces and/or protrusions. In certain embodiments, sample carriers are fabricated from UV-curable plastics, such as VeroBlue™ 3D. In other embodiments, sample carriers are fabricated from heat-set plastics, such as polypropylene or polystyrene. Sample carriers can be formed or molded as an integrated unit and, for example, may be fabricated using injection molding, machine milling, stamping, or other techniques generally known in the art. The present disclosure is not intended to be limited to any particular materials or construction methods employed with respect to sample carrier fabrication. Persons skilled in the art will recognize that many different techniques can be used to produce sample carriers of the invention. In certain embodiments, sample carriers have an ergonomic design that facilitates handling by hand.

In another aspect, sample carriers of the invention comprise an opening and a sample node, e.g., a sample node held by the opening. The opening can be any opening described herein. As used herein, a “sample node” is any substance or composite material suitable for storing biological samples in a dry state. In certain embodiments, a sample node comprises a porous substrate, such as a macroporous medium. As used herein, a “macroporous medium” is a porous substrate characterized by an average pore size greater than 1 micron. In certain embodiments, the macroporous medium has an average pore size of about 10 to about 100 microns, about 20 to about 75 microns, or about 30 to about 50 microns. In certain embodiments, a sample node comprises an open-cell foam substrate, a closed-cell foam substrate, or a combination thereof. In other embodiments, a sample node comprises an open pore substrate.

In certain embodiments, a sample node comprises a macroporous medium, wherein the macroporous medium is elastomeric. Elastomeric substrates are compressible and expandable. For example, an elastomeric substrate can be compressible to ½, ⅕, 1/10, 1/25, 1/50, or 1/100 of the volume of the uncompressed state, and expandable to 2-fold, 5-fold, 10-fold, 25-fold, 50-fold, or 100-fold the volume of the compressed state. In general, suitable elastomeric substrates are strong, possess elastic resilience, and have relatively inert surface characteristics (i.e., are relatively inert with respect to biological molecules). In certain embodiments, suitable elastomeric substrates comprise a material selected from the group consisting of polyurethane, polyvinyl alcohol, chitosen sponge, cellulose, polyester, and polystyrene. Elastomeric substrates have been described, for example, in U.S. Patent Application No. 2006/0014177.

In certain embodiments, a sample node comprises a macroporous medium, wherein the macroporous medium is non-elastomeric. Non-elastomeric substrates are essentially non-compressible and non-expandable. For example, papers (e.g., cellulose-based papers, such as filter paper) and polymer-based membranes (e.g., nitrocellulose and membranes comprising polymers such as polyesters, polyamides, etc.) are essentially non-compressible and non-expandable. Thus, in certain embodiments, a sample node comprises a cellulose-based paper. In other embodiments, a sample node comprises a polymer-based membrane. Non-elastomeric substrates have been described, for example, in U.S. Patent Application No. 2006/0014177 and PCT Application WO 03/020294.

Sample nodes suitable for use in the sample carriers of the invention can have a wide range of shapes and sizes. In certain embodiments, a sample node comprises a flat substrate (e.g., a paper or polymer-based membrane) that has been folded. In certain embodiments, the flat substrate is folded into a cup-like shape that can be held in an opening of a sample carrier.

In certain embodiments, a sample node has a spherical, elipsoidal, rectangular, cylindrical, or columnar shape (e.g., space-filling shape) that can be held in an opening of a sample carrier. In certain embodiments, a sample node has a columnar shape that is circular or polyhedral in cross-section. In certain embodiments, a sample node comprises a cavity. In certain embodiments, a sample node comprises a cavity that extends through the sample node. For example, in certain embodiments, a sample node has a cylindrical shape with a cylindrical cavity extending through it such that the overall shape is pipe-like. Without intending to be limited by theory, Applicants believe that a tube-like sample node has a larger surface area as compared to, e.g., a cylindrical sample node, thereby allowing a sample applied thereto to be absorbed more quickly and allowing a sample absorbed thereto to dry more quickly. The increased rates of absorption and drying are believed to improve the quality of the dried sample.

In certain embodiments, a sample node has a volume (e.g., a non-compressed, dry volume) of about 125 mm³, about 150 mm³, about 175 mm³, about 200 mm³, about 300 mm³, about 400 mm³, about 500 mm³, about 600 mm³, about 700 mm³, about 800 mm³, about 900 mm³, about 1000 mm³, about 1100 mm³, about 1200 mm³, about 1300 mm³, about 1400 mm³, about 1500 mm³, or more. In certain embodiments, a sample node has a surface area (e.g., a non-compressed, dry surface area) of about 140 mm², about 160 mm², about 180 mm², about 200 mm², about 220 mm², about 240 mm², about 260 mm², about 280 mm², about 300 mm², about 320 mm², about 340 mm², about 360 mm², about 380 mm², about 400 mm², about 420 mm², about 440 mm², about 460 mm², about 480 mm², about 500 mm², about 520 mm², about 540 mm², about 560 mm², about 580 mm², about 600 mm², about 620 mm², or more.

In certain embodiments, the surface area of a sample node is sufficiently large, as compared to the volume of the sample node, to allow for rapid drying of a sample applied thereto. For example, in certain embodiments, the surface area to volume ratio is at least 0.30 mm⁻¹, 0.35 mm⁻¹, 0.40 mm⁻¹, 0.45 mm⁻¹, 0.50 mm⁻¹, 0.55 mm⁻¹, 0.60 mm⁻¹, 0.65 mm⁻¹, 0.70 mm⁻¹, 0.75 mm⁻¹, 0.80 mm⁻¹, 0.85 mm⁻¹, 0.90 mm⁻¹, 0.95 mm⁻¹, 1.00 mm⁻¹, 1.05 mm⁻¹, 1.10 mm⁻¹, 1.15 mm⁻¹, or greater. Thus, for example, a sample node can have a surface area of about 145 mm² to about 175 mm², and a corresponding volume of about 135 mm³ to about 165 mm³; a sample node can have a surface area of about 375 mm² to about 455 mm², and a corresponding volume of about 510 mm³ to about 620 mm³; a sample node can have a surface area of about 540 mm² to about 660 mm², and a corresponding volume of about 1000 mm³ to about 1250 mm³; etc. More generally, a sample node can have a volume of about 150 mm³, about 200 mm³, about 300 mm³, about 400 mm³, about 500 mm³, about 600 mm³, about 700 mm³, about 800 mm³, about 900 mm³, about 1000 mm³, about 1100 mm³, about 1200 mm³, about 1300 mm³, about 1400 mm³, about 1500 mm³, or more, and a corresponding surface area that provides a surface area to volume ratio in the range of about 0.30 mm⁻¹ to about 1.15 mm⁻¹, about 0.50 mm⁻¹ to about 1.10 mm⁻¹, about 0.70 mm⁻¹ to about 1.05 mm⁻¹, or about 0.90 mm⁻¹ to about 1.00 mm⁻¹.

In certain embodiments, a sample node has a fluid holding capacity of at least about 150 μl, about 175 μl, about 200 μl, about 250 μl, about 300 μl, about 350 μl, about 400 μl, about 450 μl, about 500 μl, about 550 μl, about 600 μl, about 650 μl, about 700 μl, about 750 μl, about 800 μl, about 850 μl, about 900 μl, about 950 μl, about 1000 μl, about 1100 μl, about 1200 μl, about 1300 μl, about 1400 μl, about 1500 μl, about 1600 μl, about 1700 μl, about 1800 μl, about 1900 μl, about 2000 μl, or more.

The present disclosure is not intended to be limited to any particular sample node size or shape. Persons skilled in the art will recognize that many different sample node sizes and shapes (either folded or space-filling, with or without cavities), can be used as part of the invention. For example, in certain embodiments, a sample node is designed to be held individually by a single opening in a sample carrier. Alternatively, in certain embodiments, a plurality of sample nodes are designed to be held as a group by a single opening in a sample carrier. For example, an opening can be configured to hold a cylindrical sample node or a series of disc-shaped sample nodes that stack upon one another to form a composite object similar to the cylindrical sample node.

In certain embodiments, a sample node that is held by an opening in a sample carrier has at least 60%, 65%, 70%, 75%, 80%, 85%, 90%, 95%, or more of its surface exposed to air, e.g., not contacting a surface of the sample carrier, such as a side surface of an opening, a protrusion that extends into the opening, or a bottom surface of an opening.

In certain embodiments, a sample node has been treated with or comprises a stabilizer. As used herein, a “stabilizer” is any agent capable of protecting at least one type of biomolecule from damage during storage. In certain embodiments, the stabilizer is capable of inhibiting protein denaturation and/or undesirable contact between biomolecules and various contaminants or potential sources of degradation, including but not limited to oxygen (e.g., reactive oxygen species, such as singlet oxygen, hydroxyl radicals, superoxide anions, etc.), free water, enzymes, other reactive chemical species, and microorganisms. In certain embodiments, the at least one type of biomolecule is DNA, protein, carbohydrates, lipids, or any combination thereof.

In certain embodiments, a stabilizer comprises a filler, a reactive oxygen scavenger (ROS), a chelator, a weak detergent or emulsifier, a strong detergent, a buffer, or any combination thereof. As used herein, a “filler” is a chemical molecule that comprises a plurality of hydroxyl groups and is substantially uncharged. In certain embodiments, the filler contains no functional groups other than hydroxyl groups. In certain embodiments, the filler is extremely hydrophilic and promotes wetting of a sample node when a fluid sample is applied to the sample node. In certain embodiments, the filler also functions as a ROS. In certain embodiments, the filler is nonreactive in standard molecular and biochemical assays, such as PCR, microarrays, immunoassays, etc. Examples of suitable fillers include, but are not limited to, sucrose, mannose, trehalose, ficoll, and polyvinyl alcohol.

Examples of suitable ROSs include, but are not limited to, pyruvate, alkyl imidazoles (e.g., histidine, L-carnosine, histamine, imidazole 4-acetic acid), indoles (e.g., tryptophan and derivatives thereof, such as N-acetyl-5-methoxytryptamine, N-acetylserotonin, 6-methoxy-1,2,3,4-tetrahydro-beta-carboline), phenolic compounds (e.g., tyrosine and derivatives thereof), aromatic acids (e.g., ascorbate, salicylic acid, and derivatives thereof), azide salts (e.g., sodium azide), tocopherol and related vitamin E derivatives, and carotene and related vitamin A derivatives. Examples of suitable metal chelators include, but are not limited to, EDTA, EGTA, and o-phenanthroline. Metal specific chelators, such as copper- or iron-specific chelators, are also suitable. Examples of suitable weak detergents/emulsifiers include, but are not limited to, NP40 and Tween20. Examples of suitable strong detergents include, but are not limited to SDS and sodium lauroyl sarcosyl.

Suitable buffers can have a mildly acidic pH (e.g., about 4.0 to about 6.0) or a near neutral to slightly basic pH (e.g., about 6.5 to about 8.5). Examples of suitable buffers include, but are not limited to, Tris/HCl (pH 7-8), Tris/Borate (pH 7-8), Tris/Acetate (pH 7.8), NaAcetate (pH 4-6), citrate (pH 4-6), and boric acid (pH 4-6).

In certain embodiments, the stabilizer is added to the sample node and the sample node is allowed to dry before sample is applied to the sample node. For example, in certain embodiments, the concentration of filler in the stabilizer is selected such that, once a liquid sample is added to the sample node (e.g., an amount of liquid sample equivalent to the holding capacity/volume of the sample node), the final concentration of filler in the sample will be about 5% to about 30%, about 10% to about 25%, or about 15% to about 20%. In certain embodiments, the concentration of ROS in the stabilizer is selected such that, once a liquid sample is added to the sample node (e.g., an amount of liquid sample equivalent to the holding capacity/volume of the sample node), the final mass density of ROS in the freshly applied fluid sample will be about 10% to 30% by mass of the total specimen. In certain embodiments, the concentration of chelator in the stabilizer is selected such that, once a liquid sample is added to the sample node (e.g., an amount of liquid sample equivalent to the holding capacity/volume of the sample node), the final concentration of chelator in the sample will be about 0.1 mM to about 2 mM, about 0.5 mM to about 1.5 mM, or about 1.0 mM. In certain embodiments, the concentration of weak detergent/emulsifier in the stabilizer is selected such that, once a liquid sample is added to the sample node (e.g., an amount of liquid sample equivalent to the holding capacity/volume of the sample node), the final concentration of weak detergent/emulsifier in the sample will be about 0.5% to about 2.0%, about 0.75% to about 1.5%, or about 1.0%. In certain embodiments, the concentration of strong detergent in the stabilizer is selected such that, once a liquid sample is added to the sample node (e.g., an amount of liquid sample equivalent to the holding capacity/volume of the sample node), the final concentration of strong detergent in the sample will be about 0.1% to about 2%, about 0.5% to about 1.5%, or about 1.0%. In certain embodiments, the concentration of buffer in the stabilizer is selected such that, once a liquid sample is added to the sample node (e.g., an amount of liquid sample equivalent to the holding capacity/volume of the sample node), the final concentration of buffer in the sample will be about 10 mM to about 300 mM, with a pH as indicated above.

In certain embodiments, the stabilizer is selected to facilitate the storage and recovery of specific types of molecules, such as proteins or nucleic acids, from particular types of samples. For example, in certain embodiments, protein folding and recovery from serum or plasma is facilitated by using a stabilizer that comprises about 10% to about 20% sucrose or trehalose, about 100 mM Tris/HCl, about 1 mM EDTA, pH8. In certain embodiments, whole blood storage is facilitated by using a stabilizer that comprises about 10% to about 20% sucrose or trehalose, about 100 mM Borate, about 1 mM EDTA, about 1% NP40, pH8.

In certain embodiments, the stabilizer is selected to help sterilize a sample, e.g., by killing animal viruses (e.g., foot and mouth disease virus) and microorganisms (e.g., mold and bacteria). For example, a mildly acidic pH can be used to kill certain viruses, notably foot and mouth disease virus, while keeping nucleic acid, protein, and small molecules intact for molecular analysis. Alternatively, strong detergent can be used to kill human viruses and microorganisms (e.g., mold and bacteria) while keeping nucleic acid molecules intact for molecular analysis. Thus, in certain embodiments, nucleic acid recovery from whole blood is facilitated by using a stabilizer comprising about 20% sucrose, about 100 mM Borate, about 1 mM EDTA, about 1% SDS, pH8. In other embodiments, nucleic acid recovery from whole blood is facilitated by using a stabilizer comprising about 20% sucrose, about 1 mM EDTA, about 50 mM Na₃Citrate, about 50 mM Citric Acid, about 100 mM Boric acid, about 1% NP40, pH 5. In other embodiments, protein recovery from whole blood, plasma, or serum is facilitated by using a stabilizer comprising about 1 mM EDTA, about 50 mM Na₃Citrate, about 50 mM Citric Acid, about 100 mM Boric acid, about 1% NP40, pH 5.

In certain embodiments, a sample node comprises an identifying indicia. An “identifying indicia,” as used herein in reference to a sample node, can be any identification mechanism or means that is suitable to be used with a sample node. For example, an identifying indicia can be an identifying or detectable marker, device, signal, label, indication, output, code, etc. In certain embodiments, a sample node comprises a coding composition of detectable biological molecules. In certain embodiments, a sample node comprises a coding composition comprising a mixture of oligonucleotides, e.g., a mixture of oligonucleotides from a predetermined pool of oligonucleotides, wherein the presence or absence of oligonucleotides from the predetermined pool is indicative of a code. Suitable coding compositions have been disclosed, for example, in U.S. Patent Application No. 2005/0026181 and related U.S. patent application Ser. No. 12/471,321, filed May 22, 2009.

In certain embodiments, a sample carrier further comprises a plurality of openings and a plurality of sample nodes, wherein each sample node is held by an opening (e.g., a single opening). In certain embodiments, the openings and sample nodes are any openings and sample nodes described herein.

In another aspect, a sample carrier of the present invention comprises an opening, a sample node, and a biological sample. For example, the biological sample can be contained in a sample node of the present invention, and the sample node can be held by an opening in the sample carrier. The sample node and opening can be any sample node and opening described herein. As used herein, a “biological sample” can be any sample containing biological material(s) or molecule(s). Exemplary biological samples include any primary, intermediate or semi-processed, or processed biological samples, e.g., blood, serum, plasma, urine, saliva, spinal fluid, cerebrospinal fluid, milk, or any other biological fluid, skin cells, cell or tissue samples, biopsied cells or tissue, sputum, mucus, hair, stool, semen, buccal samples, nasal swab samples, or homogenized animal or plant tissues as well as cells, bacteria, virus, yeast, and mycoplasma, optionally isolated or purified, cell lysate, nuclear extract, nucleic acid extract, protein extract, cytoplasmic extract, etc. Biological samples can also include, e.g., environmental samples or food samples, to be tested for microorganisms.

Exemplary biological samples also include any composition or material containing biomolecule(s), either naturally existing or synthesized, e.g., DNA, RNA, nucleic acid, polynucleotide, oligonucleotide, amino acid, peptide, polypeptide, biological analytes, drugs, therapeutic agents, hormones, cytokines, etc. The biological samples can be provided fresh, such as blood samples obtained from a finger stick or a heel stick and directly applied to a sample node. Alternatively, the biological samples can be provided in a container or via a carrier. In certain embodiments, a biological sample is pretreated or partially treated, e.g., with a lysing agent, such as a detergent (e.g., SDS or Sarcosyl), a precipitating agent, such as perchloric acid, a chaotrope, such as guanidinium chloride, a precipitating agent, such as acetone or an alcohol, or some other agent. In certain embodiments, a biological sample is absorbed to, or stored or maintained in a sample node, e.g., dry storage of a biological sample in a sample node.

In certain embodiments, the sample carrier of the present invention further comprises a plurality of openings, at least one sample node, and at least one biological sample, wherein each sample node is held by an opening (e.g., a single opening), and wherein each biological sample is contained in a sample node (e.g., a single, discrete sample node).

In another aspect, storage systems are provided. The storage systems can comprise the sample carrier of the present invention and a receptacle. As used herein, a “receptacle” can be any container that interfaces with the sample carrier. In certain embodiments, the receptacle holds the sample carrier. For example, in certain embodiments, a receptacle is a tray. In certain embodiments, a receptacle is a tray that includes one or more slots in which a sample carrier can be lodged, e.g., for storage. In certain embodiments, a receptacle is able to hold 1, 2, 3, 4, 5, 6, or more sample carriers. In certain embodiments, a receptacle is a tray that further comprises a cover. In certain embodiments, a receptacle has a standard SBS microplate footprint, e.g., a 127.76 mm×85.47 mm footprint.

In certain embodiments, the receptacle interfaces and thereby seals the sample carrier. For example, in certain embodiments, a receptacle comprises a sealing mechanism configured to engage a sample carrier, thereby creating a sealed chamber around the opening for the sample node and any sample node held thereby. The sealing mechanism can be, for example, a screw mechanism, such as threading (e.g., capable of interlocking with threading on the sample carrier), or a friction-based locking mechanism, such as a groove or notch designed to receive a lip or hook located on the sample carrier, or vice versa. In certain embodiments, a receptacle comprises a drying agent, such as a desiccant (e.g., silica or dryerite, or the equivalent).

In certain embodiments, a receptacle comprises an identifying indicia. An “identifying indicia,” as used herein in the context of a receptacle, is anything that helps to identify the receptacle and/or any sample carriers or sample nodes stored within the receptacle. Examples of such identifying indicia include, but are not limited to, hand-written information (e.g., patient identification information), a label (e.g., a typed or computer-generated label), a bar code (e.g., a bar code that can be read by an optical scanner), a transceiver (e.g., a transceiver that is responsive to a query signal and emits an identification signal in response to the query signal), and/or a biological coding composition. Suitable biological coding compositions are disclosed, for example, in U.S. Patent Application No. 2005/0026181. The present disclosure is not intended to be limited to any particular identifying indicia. Persons skilled in the art will recognize that many different identifying indicia can be used in conjunction with receptacles of the invention, depending upon its intended use.

In certain embodiments, a storage system further comprises a plurality of sample carriers and one or more receptacles, wherein each receptacle is suitable for storing one or more sample carriers.

In another aspect, the present invention provides methods of collecting, shipping, and/or storing biological samples, e.g., by using a sample carrier of the present invention and, optionally, a corresponding receptacle. For example, methods of collecting a biological sample can comprise applying a biological sample to a sample node held by an opening in a sample carrier of the present invention. The collection can be direct (i.e., the sample is transferred to directly to the sample node via contact with a subject or specimen) or indirect (e.g., collection of the sample occurs separately from the sample being applied to the sample node).

In certain embodiments, the methods comprise drying a sample node that a biological sample has been applied to. The drying can be facilitated or not. In certain embodiments, facilitated drying comprises drying the sample node to which the biological sample was applied in a low humidity chamber, such as a chamber having a humidity level of 35%, 30%, 25%, 20%, 15%, 10%, 5%, or less. In certain embodiments, facilitated drying comprises circulating air around the sample node as it is drying, e.g., using a fan. In certain embodiments, facilitated drying comprises drying the sample node to which the biological sample was applied in a low humidity chamber, wherein the air within the chamber is being circulated. In certain embodiments, facilitated drying comprises sealing the sample carrier with a corresponding receptacle, wherein the receptacle comprises a desiccant (e.g., silica, dryerite, etc.).

In certain embodiments, the methods of collecting, shipping, and/or storing a biological sample can comprise sterilizing a sample carrier that comprises a sample. Sterilization (e.g., of the external surface of a sample carrier) can kill or prevent the spread of infectious agents associated with a sample stored in the sample carrier. Sterilization can be performed chemically. For example, an acid (e.g., having a pH of about 5.0 or less, such as vinegar) can be used to kill infectious agents that are acid sensitive, such as foot and mouth disease virus. Alternatively, an alcohol or other organic sterilizer can be used to kill infectious agents such as human viruses. Liquid sterilizers can be sprayed onto a sample carrier and then wiped off, or wipes comprising the liquid sterilizer can be used. Radiation, such as beta radiation or UV radiation, can also be used to sterilize sample carriers, providing that the sample carriers are made from materials that are not penetrated by such forms of radiation.

In certain embodiments, a sample carrier is sealed with a corresponding receptacle prior to external sterilization. In certain embodiments, the seal is air-tight and/or impermeable to liquid sterilizers. Sealing and sterilizing a sample carrier can not only prevent the spread of infectious agents associate with the sample stored in the sample carrier, but it can also prevent the stored sample from becoming contaminated.

In certain embodiments, the methods of collecting, shipping, and/or storing biological samples further comprise recording an identifying indicia associated with the biological sample. Identifying indicia associated with a biological sample can include, for example, identifying indicia from a sample carrier (e.g., a barcode) and/or a sample node (e.g., a coding composition) that the biological sample is stored upon. Identifying indicia associated with a biological sample can be recorded on paper medium or electronic medium, such as a computer. The record thus created can be stored in a data repository, such as a file or a computer database.

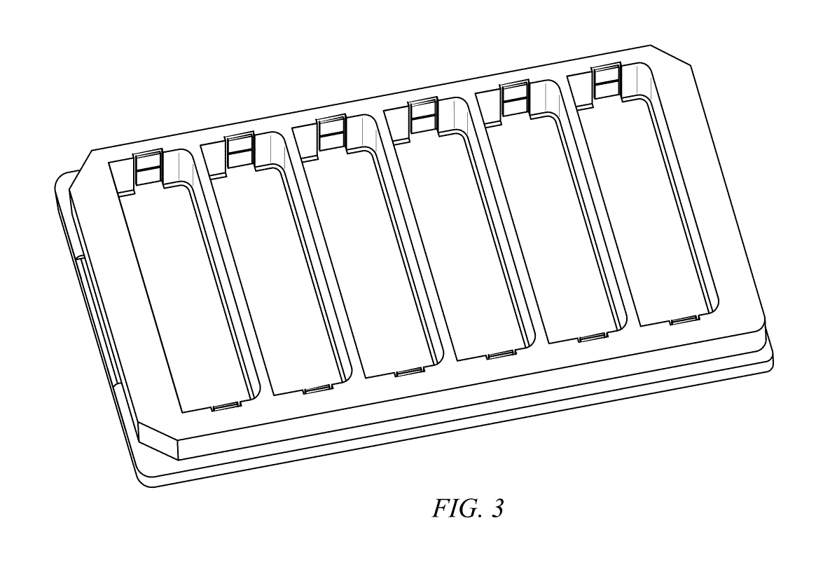

In certain embodiments, one can further store and/or seal a sample carrier that comprises a biological sample by interfacing the sample carrier with a receptacle. For example, in certain embodiments, the receptacle can be a tray that holds one or more sample carriers, such as shown in FIG. 3. Such receptacles can be placed into storage, e.g., in an archive. Archives have been described, e.g., in U.S. Pat. No. 7,142,987.

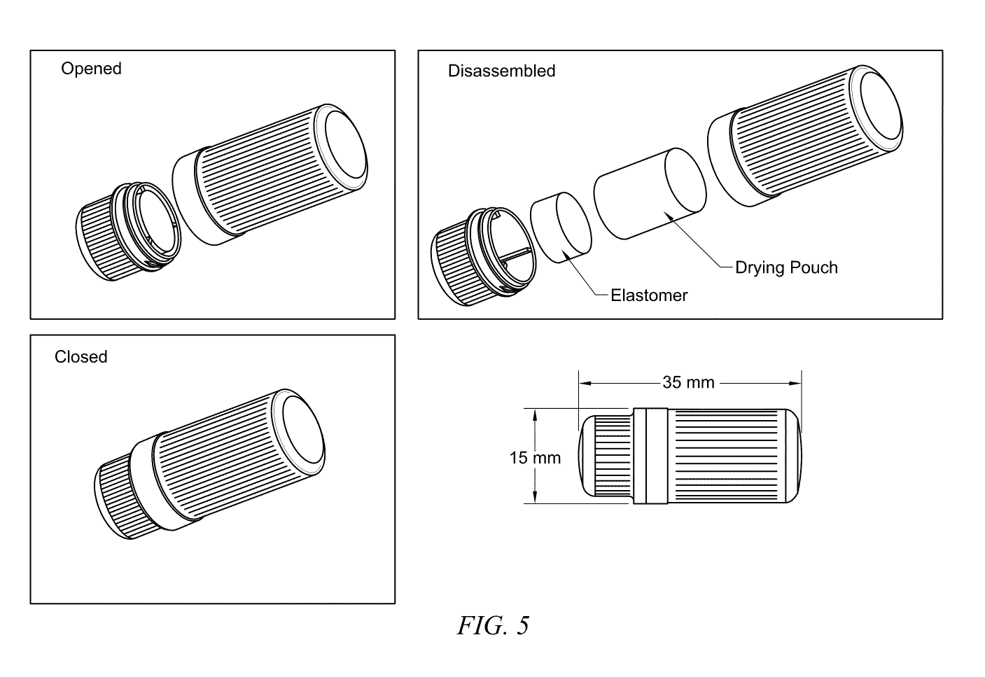

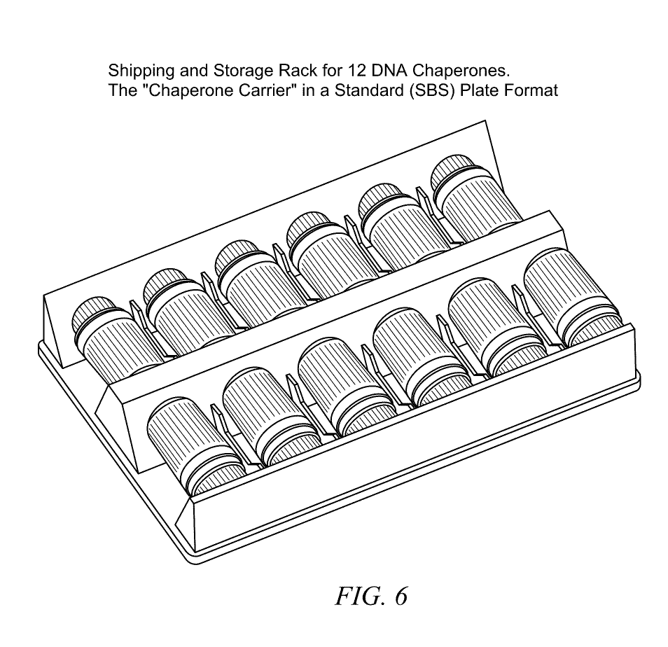

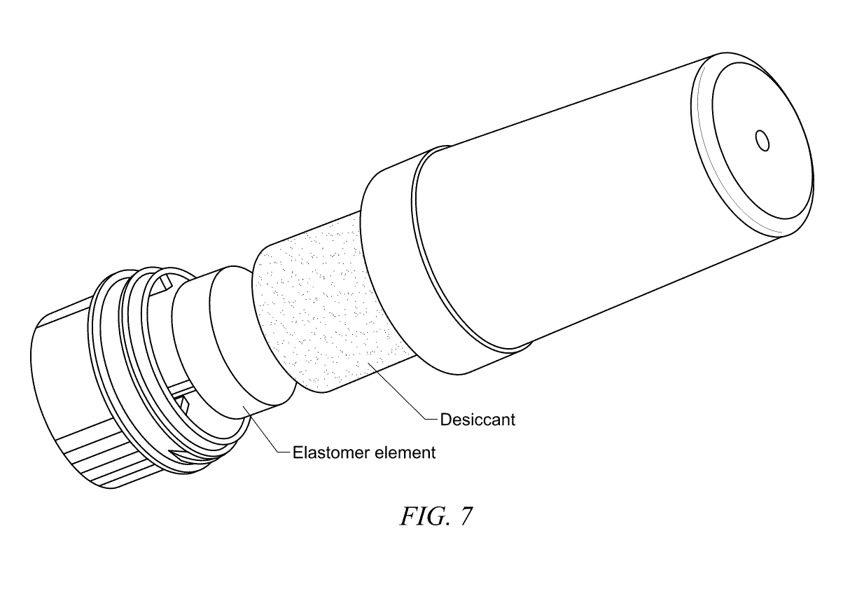

In certain embodiments, the receptacle forms a seal with the sample carrier, such as shown in FIGS. 5 and 7. In certain embodiments, the receptacle can form a seal and also hold the sample carrier. Alternatively the receptacle can form a seal with the sample carrier, and the resulting storage system can be placed in a tray for storage purposes, such as shown in FIG. 6. Such a tray can be placed into storage, e.g., in an archive. In certain embodiments, a biological sample stored in a sample carrier of the invention can be retrieved after it has been stored.

In certain embodiments, sample carriers comprising biological samples are shipped from a location where the sample is collected to another location where the sample is to be stored or processed. The shipping can comprise first sealing and/or sterilizing the sample carrier. In addition, the shipping can comprise tracking the progress of the sample carrier. For example, in certain embodiments, the sample carrier comprises identifying indicia and the identifying indicia is monitored/read when the sample passes through an intermediate location on its transport path (e.g., a shipping hub where sample carriers are collected and routed). The transport and tracking can be performed in a manner analogous to how packages are transported and tracked in standard shipping operations, such as FedEx or the USPS.

In another aspect, methods of recovering a biological sample are provided. The methods can comprise removing a sample node carrying the biological sample from the sample carrier of the present invention. Removing a sample node can comprise pushing the sample node out of an opening in the sample carrier. Alternatively, removing a sample node can comprise pulling the sample node from an opening in the sample carrier.

Methods of recovering a biological sample of the invention can further comprise rehydrating a sample node that has been removed from a sample carrier. A sample node can be rehydrated, for example, by adding a fluid, such as water or a buffer (e.g., wash buffer or rehydration buffer), to the sample node. Alternatively, a sample node can be rehydrated by adding a fluid, such as water or an appropriate buffer, to a sample carrier comprising the sample. For example, the opening in the sample carrier can have a concave topology that is capable of holding rehydrating fluid in a manner that allows the sample node held by the opening to be rehydrated. Following rehydration, the rehydrating fluid can be removed from the sample node. For example, the sample node can be compressed and/or centrifuged to remove the rehydrating fluid. The amount of rehydration fluid used to recover sample can be equal to the volume of the sample node (e.g., elastomeric sample node) that the sample is attached to or resting upon. Rehydrating fluid obtained from a sample node in this manner will typically contain molecules of interest originating from the biological sample, such as DNA, RNA, protein, lipids, hormones, small molecule analytes, drugs, and other biological molecules. Proteins that can be recovered from sample nodes of the invention include, for example, Pregnancy Associated Plasma Protein A (PAPP-A), Human Chorionic Gonadotropin (hCG), and Thyroid Stimulating Hormone (TSH), to mention just a few. Small molecules and peptides that can be recovered from sample nodes of the invention include, for example, unconjugated estriol (uE3), Interleukin 6 (IL6), and Cotinine (Cot).

Methods of recovering a biological sample of the invention can result in partial purification of protein, small molecule components, and DNA. For example, as illustrated in Example 8, a first wash buffer (e.g., a buffer having a pH of about 7.0 to about 8.5, such as a Tris-based buffer) can be used to rinse some protein and small molecule components from a sample node (e.g., comprising an elastomer substrate). Subsequently, a high pH elution buffer (e.g., 30 mM CABS, pH 10-11) can be used to recover partially purified DNA from the sample node.

In yet another aspect, kits for collecting, shipping, and/or storing biological samples are provided. In certain embodiments, the kits comprise a sample carrier of the invention. In other embodiments, the kits comprise a storage system of the invention.

The following examples illustrate sample carriers of the invention and increases in the rate of sample node drying enabled by the sample carriers. The examples should, of course, be understood to be merely illustrative of only certain embodiments of the invention and not to constitute limitations upon the scope of the invention which is defined by the claims that are appended at the end of this description.

### EXAMPLES

### Example 1

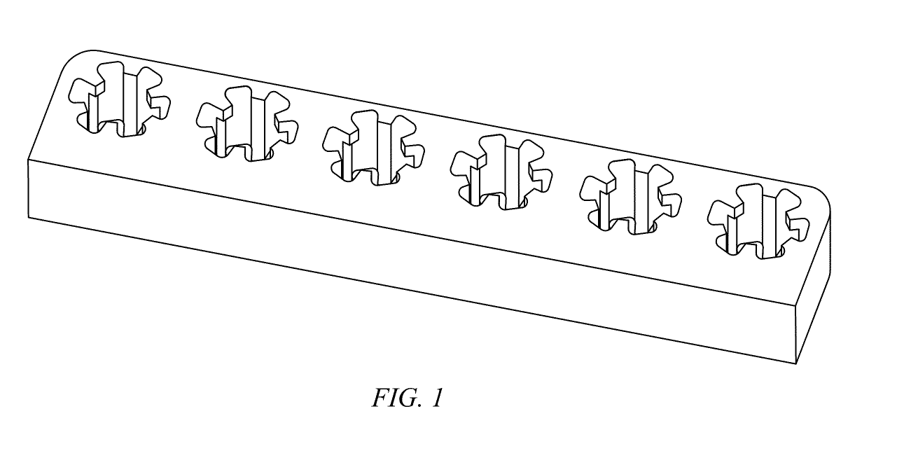

As shown in FIG. 1, a sample carrier of the invention can include six cylindrical openings that pass entirely through the sample carrier. Each opening includes six sub-opening spaces and six inwardly-pointing protrusions. Each sub-opening space has an increasingly larger width outwards of the center of the opening. The openings are asymmetrically positioned such that they are closer to the rear margin of the sample carrier, thereby providing space at the front margin of the sample carrier that can be used to hold the sample carrier and/or present an identifying indicia, such as a bar code. In this embodiment, the sample carrier is substantially flat and has dimensions of 70 mm (width)×15 mm (depth)×7 mm (thickness). The central axes of the openings are located 9.5 mm from the rear margin of the sample carrier, and are separated from one another by 11.6 mm.

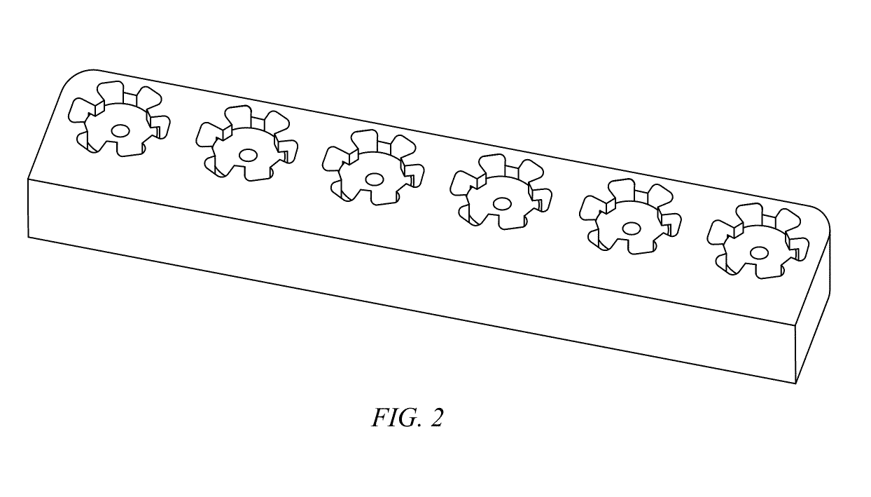

As shown in FIG. 2, each opening of the sample carrier of FIG. 1 is capable of holding a sample node. In this embodiment, the sample nodes are cylindrical, have dimensions of 6 mm (diameter)×5 mm (height), and include a central cavity extending through the sample node. The inwardly-pointing protrusions of the openings contact, and thereby hold the sample nodes. In the embodiment of FIG. 2, three alternate inwardly-pointing protrusions include a lip at their top end such that the sample node is held by a combination of pressure contacts and frictional resistance provided by the lip. The lip could similarly be located at the bottom end of the protrusions. In fact, in this embodiment, the other three inwardly-pointing protrusions include a lip at their bottom ends, such that the six protrusions provide lips positioned at the top and bottom end of the sample node that help to hold the sample node in place.

Upon application of a fluid sample to a sample node and subsequent air-drying, a sample carrier holding the sample node can be sealed using a film, such as a laminating plastic. The film can, for example, be placed over the top and bottom face of the opening in the sample carrier, thus sealing the sample node and any biological sample attached thereto from additional contact with the outside.

As shown in FIG. 3, a receptacle of the invention can include six slots designed to hold sample carriers of the type shown in FIG. 1. In this embodiment, the receptacle has a flat, plate-like structure having dimensions of 127.76 mm×85.47 mm. The design of the receptacle allows for a cover or lid to be placed on top. The cover can be a film laminate or a reversibly positioned lid.

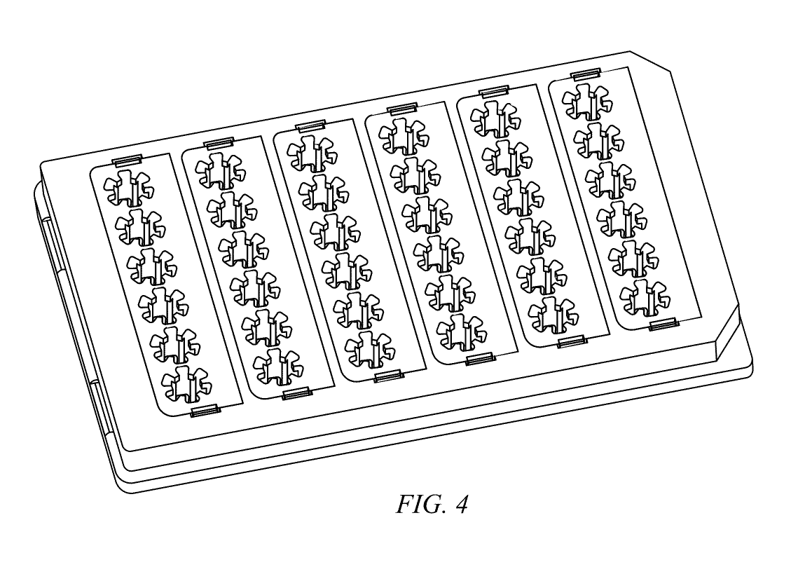

As shown in FIG. 4, a storage system of the invention can comprise a receptacle of the type shown in FIG. 3 and a plurality (in this case 6) of the sample carriers of FIG. 1 inserted therein.

### Example 2

As shown in FIG. 5, a storage system of the invention can include a sample carrier comprising a cup-like morphology and a corresponding receptacle. In this embodiment, the sample carrier comprises an opening and a sample node, wherein the opening has three ridge-like protrusions that hold the cylindrical, 6 mm (diameter)×5 mm (height) elastomeric sample node. The sample carrier also includes a sealing mechanism—threading—which can interface with threading on the corresponding receptacle and thereby seal the sample carrier, protecting the sample node from external contamination and containing any infectious agents associated with a sample stored on the sample node. In this embodiment, the receptacle comprises a drier packet (e.g., comprising a desiccant) capable of driving evaporation of water from a sample applied to the sample node of the sample carrier.

As shown in FIG. 6, a tray of the invention can be used to hold storage systems. In this embodiment, the tray holds up to 12 storage systems of the type shown in FIG. 5. The tray has a standard SBS plate format, amenable for use, for example, in existing archive systems.

FIG. 7 shows another embodiment of a storage system of the invention. This embodiment is highly analogous to the embodiment shown in FIG. 5, but has been scaled up to hold a 12 mm (diameter)×5 mm (height) elastomeric sample node. The increased size of the sample node allows for collection of larger volume specimens, such as: blood samples from an ear piercing (livestock & other animals); blood samples from a heal stick (humans, especially neonates); blood samples from a finger stick (humans); sputum collected directly from the mouth (humans); urine (humans & other animals); and milk collected directly from contact with the utter or by pipetting (livestock & other animals).

### Example 3

The rate of sample node drying was evaluated for sample carriers of the type shown in FIG. 2. Two sample carriers with six 150 uL polyurethane sponge sample nodes were first weighed without any sample. Then, 150 μL of 100 mM Tris Buffer or 150 μL of whole blood was added to each of the six wells in one of the sample carriers. The samples were allowed to soak into the elastomer sample node, the entire sample carrier was weighed again, and the weight of the sample carrier without any sample was subtracted to generate the “0” time point. While drying, the sample carriers were stored in a chamber at regulated humidity (35%) which had, within it, a small fan to circulate air around the sample carriers. The sample carriers were removed from the chamber over time and re-weighed to determine the rate of evaporative water loss from the elastomer sample nodes. As shown in Table 1, the time of half maximal evaporative loss occurred at less than 1 hour for both sample types, with complete dryness obtained after one hour for Tris buffer and after three hours for blood. Blood appears to dry more slowly than Tris buffer and to generate a dried product with residual solid mass comprising 0.15/1.25=12% of the total blood fluid mass. Blood is known to be 12%-15% solids by weight, in good agreement with the amount of dried residue measured in Table 1.

TABLE 1

| | Tris Buffer, | Blood, |
|---|---|---|
| Drying Time | Sample Fluid | Sample Fluid |
| (hours) | Weight (Grams) | Weight (Grams) |
| 0 | 1.15 | 1.25 |
| 1 | 0.10 | 0.45 |
| 2 | 0.00 | 0.35 |
| 3 | 0.00 | 0.15 |
| 4 | N/D | 0.15 |

For comparison, the rate of sample node drying was evaluated for 6 mm×5 mm polyurethane sponge sample nodes held in cylindrical, 6 mm diameter×10 mm deep, flat-bottom microplate wells in strip plates having six such wells. The wells lacked ventilation spaces and sub-opening spaces because there was no air gap between the elastomer sample nodes and the wells. Strip plates, each with six 150 uL elastomer sample nodes, were first weighed without any sample. Then, 150 uL of 100 mM Tris buffer sample was added to each of the six wells in a plate, allowed to soak into the elastomer sample node, the entire strip plates, plus sample, was weighed again, and the weight of the strip plates prior to addition of sample was subtracted to generate the “0” time point. After initial weighing, the strip plates were stored in a chamber at regulated humidity (either 20% RH or 35% RH). The strip plates were removed from the chamber from time-to-time and re-weighed to determine the rate of evaporative water loss from the elastomer sample node. As seen in Table 2, at both relative humidity values the time of half maximal evaporative loss occurred at around 10 hours, with complete dryness obtained between 22 and 46 hours.

TABLE 2

| | Tris Buffer at 20% RH, | Tris Buffer at 35% RH, |
|---|---|---|
| Drying Time | Sample Fluid | Sample Fluid |
| (hours) | Weight (Grams) | Weight (Grams) |
| 0 | 1.126 | 1.116 |
| 1.5 | 1.103 | 1.038 |
| 3 | 0.920 | 0.963 |
| 4 | 0.838 | 0.913 |
| 22 | 0.166 | 0.307 |
| 46 | −0.007 | 0.004 |
| 70 | −0.008 | 0.003 |

The data in Tables 1 and 2 are presented as the net increase in fluid weight due to sample addition, as a function of drying time.

The drying kinetics shown in Tables 1 and 2 demonstrate about a 10-fold increase in drying rate achieved with a elastomer sample node held in the sample carrier of FIG. 2 relative to identical 150 uL elastomer sample nodes held in a cylindrical flat-bottom microplate well that lacks ventilation or sub-opening spaces. Without intending to be limited by theory, the large evaporative rate increase seen in the sample carriers of FIG. 2 is attributed to the increase in elastomer sample node surface area directly exposed to air and to the fact that, in a drying chamber with induced air flow, the sub-opening spaces allow laminar air flow around the surfaces of the sample nodes, even while they are held within the sample carrier opening, thus additionally increasing the drying rate.

### Example 4

Drying of whole blood on a 6 mm×5 mm cylindrical elastomer was measured inside a sample carrier identical to that described in FIG. 5. The carrier comprises a cup-like topology which holds a single 6 mm×5 mm elastomer sponge and a sealing mechanism featuring threading designed to interface with the threading on a corresponding receptacle. The sample carrier is designed to hold a single sample node via three ridge-like protrusions, with ventilation spaces created in the space bounded by the protrusions, the side surface of the opening and the side surface of the sample node. The corresponding receptacle is designed to hold a silicon desiccant to facilitate drying of the sample node after the sample carrier is sealed with the receptacle.

In this example, the weight of the carrier plus the elastomer sponge was measured at time zero. The weight was then re-measured after addition of a fluid stabilizer comprising:

A) 20% sucrose (as a filler and ROS scavenger)

B) 1 mM EDTA (as a metal chelator and inhibitor of microbial growth)

C) 1% NP40 (as an emulsifier and inhibitor of microbial growth)

The weight of the carrier plus elastomer plus added stabilizer was then remeasured after the stabilizer had been allowed to dry to completion in the open air. At that time, 100 μL of fluid human blood was added to the elastomer plus dried stabilizer in the carrier. Its weight was re-measured and then the carrier with added blood was connected to the corresponding receptacle, bearing a silicon drier pack, in order to initiate blood drying inside the sealed assembly.

After 24 hours, the carrier was temporarily separated from the receptacle and the weight of the carrier+elastomer+blood specimen was re-measured. After re-measurement, the carrier was re-connected to the receptacle and drying was continued for an additional 24 hours. The result of these measurements is shown in Table 3, which shows that, upon 24 hours of drying inside the sealed carrier-receptacle assembly, approximately 76% of the initial blood weight had been lost by evaporation by transfer to the enclosed drier pack. Continuation of the drying process for an additional 24 hours produced only an additional 2% of weight loss, thus demonstrating that the majority of all evaporative water loss from the blood specimen had been incurred during the first 24 hours.

By reference to data as in Tables 1 & 2, the data of Table 3 demonstrates that blood applied to a 6 mm×5 mm elastomer (treated with stabilizer) proceeds to dryness inside the type of closed carrier+receptacle assembly displayed in FIG. 5.

TABLE 3

| | Time point | Wt |
|---|---|---|
| Weight (Wt) measurements | (hr) | (g) |
| Wt of carrier alone | 0 | 1.146 |
| Wt of carrier + elastomer sponge | 0 | 1.172 |
| Wt of carrier + sponge + dried | 0 | 1.199 |
| stabilizer | | |
| Wt of carrier + sponge + stabilizer + | 0 | 1.299 |
| 100 ul fluid blood | | |
| Net wt of added 100 ul fluid blood | 0 | 0.100 |
| Wt of carrier + sponge + stabilizer + | 24 | 1.213 |
| 100 ul fluid blood after 24 hrs of | | |
| drying | | |
| Wt of carrier + sponge + stabilizer + | 48 | 1.211 |
| 100 ul fluid blood after 48 hrs of | | |
| drying | | |
| Wt lost from sponge after 24 hours | 24 | 0.076 |
| of drying | | |
| Residual wt of dried sponge + | 24 | 0.024 |
| stabilizer + dried blood after 24 hrs | | |
| Wt lost from sponge after 48 hours | 48 | 0.078 |
| of drying | | |
| Residual wt of dried sponge + | 48 | 0.012 |
| stabilizer + dried blood after 48 hrs | | |
| Fractional blood weight remaining | 24 | 24% |
| after 24 hours of drying | | |
| Fractional blood weight remaining | 48 | 22% |
| after 48 hours of drying | | |

### Example 5

In general, it has been observed that when a 150 μl-capacity 6 mm×5 mm elastomer sample node is loaded with 150 μl of whole blood and allowed to air dry in a sample opening which lacks sub-opening spaces, the drying process requires approximately 24 hours to complete at room temperature. It has also been observed that the same elastomer sample node loaded with a 150 μL fluid blood sample will evaporate to dryness in less than 24 hours in a sample carrier comprising sub-opening spaces and having the representative design shown in FIG. 2 or the enclosed tube design as in FIG. 5.

Enhancement of drying rate has utility at a minimum of 4 levels of practical concern.

1) Processing speed—A 24 hour drying rate provides for a processing bottleneck under conditions when many samples must be collected at once. Thus, an enhancement of drying rate is of logistical value in lab work-flow.

2) Microbial contamination—Biological samples stored on or in a sample node are at maximum biological risk during the drying process, in the transitional period where the sample is at room temperature but remains in the fluid phase. During that period, there is opportunity for the fluid sample to be contaminated with yeast, mold and bacteria, and to incur microbial growth upon the sample. By enhancing the rate of sample drying, the specimen in a node quickly assumes the air-dried state, which is more resistant to airborne contamination than is the case for a fluid sample. Upon drying, samples in a node become incompatible with microbial growth, which generally requires a sample to be well hydrated.

3) Biochemical degradation by Hydrolysis—The enzymes which catalyze the degradation of protein and nucleic acids have significant activity at room temperature in a fluid biomolecule preparation. Thus, in typical lab work flow, fluid samples must be continuously refrigerated. Dry state sample storage inhibits such enzymatic activity because such enzymes are generally inactive upon de-hydration and because the degradative chemical reactions which they catalyze typically entail the addition of water (i.e., hydrolysis) of a protein or nucleic acid molecule, thus producing protein or nucleic acid backbone cleavage. In the dry state, there is little or no water available as a chemical reactant to support such enzyme catalysis. Additionally, any non-enzymatic hydrolysis of protein or nucleic acid is similarly inhibited, since water is generally unavailable for such reactions. Integrated over time, the amount of undesired protein or nucleic acid hydrolysis will be proportional to the time the sample spends in the fluid state prior to dryness. For example, if the drying rate were increased 5-fold, the period over which the sample remained fluid would be reduced 5-fold, producing an (approximate) 5-fold decrease in the amount of enzymatic or non-enzymatic sample hydrolysis.

4) Sample Degradation by Oxidation—Proteins, drugs and other small molecules, especially lipids, are particularly unstable with respect to air-mediated oxidation at room temperature while the sample remains in the fluid phase. However, upon air-drying to a solid, the resulting solid biological sample becomes much less permeable to oxygen exposure from the air, since the oxygen from air must diffuse into the sample through the interstices of a sample node that is filled with solid sample. Generally, the diffusion rate and associated permeability of oxygen is much higher in a fluid as compared to in a solid, so once a biological sample has air-dried and the interstices of the sample node harden to a solid, the rate of oxygen mediated damage (which can only occur if oxygen permeates the sample) is greatly reduced.

### Example 6

Use of sample nodes comprising an elastomer substrate in the sample carriers of the invention is typically superior to sample nodes comprising a filter paper substrate in several important ways:

A) The three-dimensional characteristics of the elastomer result in a fluid-holding capacity that is much greater than two-dimensional filter paper. For example, a 6 mm (diameter)×5 mm (height) elastomer will hold about 150 μL of a fluid such as blood, which is approximately 10 times greater than the volume of fluid that can be sequestered within a 6 mm disc of filter paper. Expansion of the sponge dimensions to 12 mm (diameter)×10 mm (height) increases the blood storage volume to over 1 ml. At that larger volume, 1 ml of blood could be stored dry or 1 ml of a sputum or other interesting sample, allowing for dry state biospecimen storage for a large range of applications.

B) The pore structure of an elastomer is very different from that of filter paper and is more conducive to macromolecular diffusion and, hence, rapid recovery. For example, elastomers are typically made from chemical foams. Upon hardening, such foams form a smooth, open (worm-like) pore structure which results from fusion of the polymeric material from which they were formed. On the other hand, filter paper substrates, such as FTA™ or Whatman 903™ Guthrie cards are formed by mechanical compression (matting) of cellulose to form a chaotic, web-like pore structure which, by means of its irregularity, presents a “tortuous” diffusional path for the input or exit of cells or macromolecular solutes. Especially for macromolecules, such tortuousity, as defined formally in polymer physical chemistry, produces a significant barrier to the release of such macromolecules from the filter paper. Bulk diffusion of macromolecules within open-pore structures such as an elastomer is much greater, thus greatly facilitating sample efflux upon rehydration.

C) An elastomer is capable of being mechanically compressed to release its original fluid contents, with little or no final dilution. Cellulosic filter papers (such as Guthrie cards or FTA™) are, generally speaking, an incompressible medium. Thus, dried specimens are typically recovered by addition of a large excess of a hydrating fluid, followed by agitation or prolonged unstirred soaking. Thus, standard protocols for dried blood recovery from dried blood spots involve rehydration in at a ten-fold volume excess of hydration fluid, relative to the volume of the original fluid sample. Elastomers (like all ordinary utility sponges) are quite different, in the sense that the fluid contents of a sponge may be recovered by simple mechanical compression. Such compression can be induced by low speed bench-top centrifugation in a spin basket. At >1000 G, such sponges instantaneously collapse and eject the full fluid content in a way that is nicely suited to routine laboratory processing. Thus 150 μL of blood (about three drops) can be added to a 150 μL cylinder-shaped elastomer, allowed to air-dry, rehydrated in as little as 150 μL of water, then “squeezed” in a centrifuge to release the full complement of re-hydrated blood at essentially the original fluid concentration. The ability to recover a relatively large volume of dried blood in that very efficient way is a fundamental enhancement relative to the use of filter paper for blood spot collection.

D) Elastomers can be pre-treated with nearly any desired combination of stabilizing solutes. By analogy with chemically treated filter paper such as FTA™ (which is essentially an ordinary Whatman 903™ Guthrie card plus Tris, EDTA, SDS and Uric acid) an elastomer can be treated with any number of chemical solutes: to facilitate wetting of a dried blood sample; to chelate metals; to provide a detergent to disrupt nucleic acid-protein complexation; to scavenge reactive oxygen species (ROS); and to inhibit microbial growth upon the dried sample. For example, the following stabilizer provides excellent recovery of both intact, high molecular weight DNA and the recovery of a relatively large number of proteins in a state that support unaltered Luminex based Immunoassay:

A) 20% sucrose (as a filler and ROS scavenger)

B) 1 mM EDTA (as a metal chelator and inhibitor of microbial growth)

C) 1% NP40 (as an emulsifier and inhibitor of microbial growth)

This stabilizer can be added to an elastomer (e.g., a volume of stabilizer equivalent to the volume of the elastomer), and then allowed to dry to produce the treated elastomer, ready for application of blood.

At the DNA level, we have found that 150 μL of dried blood stored on an elastomer can be re-hydrated after up to 34 days of storage at RT or 56° C. (133° F.) by addition of the same standard protease solutions used to process fresh blood, followed by standard protease treatment at 56° C. and then fluid release by a one minute of centrifugation in a spin basket followed by a standard Qiagen Mini prep column.

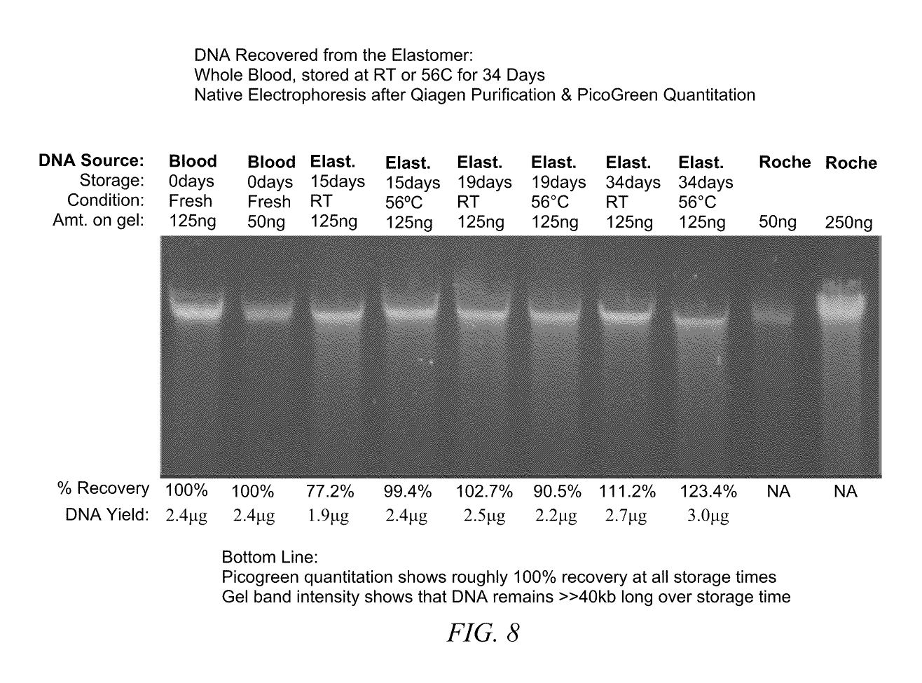

As seen in FIG. 8, when analyzed via PicoGreen fluorimetry, the DNA yield per 150 μL of dried blood input is in the 2.5 μg to 3 μg range, which corresponds to approximately 100% recovery relative to recovery from 150 μL of fresh blood starting material (see the quantitation below the 1% agarose gel image). Upon loading 125 ng of such DNA per gel lane, the ethidium-stained gel images reveal a standard collapsed band with apparent length of >40 kb, indicative of a length distribution greater than 40 kb, the maximum sieving range of such gels. Thus, as assessed by this standard gel analysis, the DNA complement of whole blood has remained very high molecular weight in the elastomer, even after prolonged dry state storage at 56° C.

We propose that, relative to traditional filter paper cards, the observed 20-fold increase in blood DNA storage capacity has the attributes of an enabling technology. At present, those interested in genome wide association studies require at least 1 μg of DNA, which cannot be obtained from filter paper. This has lead to the use of saliva collection (e.g., as via Oragene technology) which can yield several micrograms of DNA (human plus bacterial) but has proven to be costly and, based on the limited content structure of saliva, cannot be used for analytes other than DNA. Sample carriers of the invention comprising sample nodes that include an elastomer substrate can replace both filter paper and saliva as the basis for such high value (GWAS) microarray testing and possible follow-on, low cost re-sequencing technologies to come in the near future, as we approach an era of the $1000 genome.

### Example 7

This example demonstrates the storage and recovery of protein on sample carriers comprising a sample node comprising an elastomer substrate. Analytes were tested at Rules Based Medicine (Austin Tex.) in a multiplexed fashion via the Luminex-RBM bead immunoassay platform. 150 μL samples were applied to 150 μL elastomer substrates, each with a different set of chemical stabilizer treatment dried into the elastomer. The samples were then air dried at room temperature (RT, or 25° C.) for a day followed by RT storage in the air-dried state.

The samples consisted of serum (SST), EDTA-treated plasma (EDTA Pls), heparin-treated plasma (Hep Pls), Citrate-treated plasma (Cit Pls), and whole blood (WB). Upon drying, these specimens were sealed and then stored at 25° C. for 28 days. Following storage, the specimens were rehydrated by adding 130 μL of water, incubated at RT for 30 minutes, then ejected from the elastomer by spinning for 5 minutes at 1000 g in a microfuge spin basket. The recovered samples were analyzed by Rules Based Medicine (Austin Tex.) on their 150 analyte MAP screening panel, based on a highly multiplex Luminex immunoassay. Of the 114 analytes which gave non zero values in the freshly collected (never dried) starting material, the apparent protein concentration measured after drying and re-hydration was compared to the value measured for the freshly collected samples. Error bars correspond to one SD among the four subjects tested.

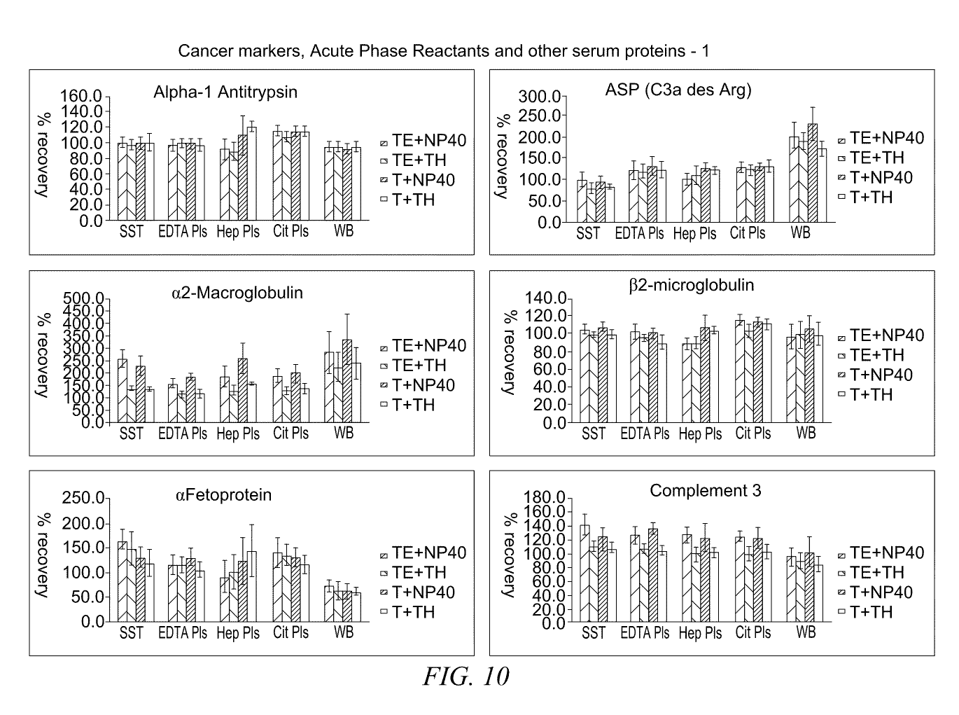

Data in FIG. 10 have been presented as percent recovery for a fraction of those 114 non-zero protein analytes, comprising the multiplex panel of the most abundant protein species. As seen from this panel, and as assessed by Luminex immunoassay, there is surprisingly little change in the apparent analyte concentration for any of the 6 protein species, relative to freshly collected plasma of whole blood. These representative data demonstrate that when stabilized via air-drying in the elastomer, the predominant serum proteins remain viable as substrate for quantitative immunoassay, over at least two months of RT storage.

### Example 8

Direct sample collection, as well as sample purification, is facilitated by the sample carriers of the present invention. For example, following a finger prick, blood can be directly transferred by passive wicking from the finger to a sample node held by a sample carrier. The blood sample is then allowed to dry on the sample node, either passively or in a facilitated manner, such as when the sample carrier is sealed with a corresponding receptacle that includes a desiccant. Following transfer of the sample, an identifying indicia associated with the sample carrier (e.g., a bar code or radio-frequency tag) can be recorded, thereby linking a specific sample with a specific sample carrier. The foregoing procedure is amenable to not only the collection of blood, but the collection of many other types of samples as well, such as sputum, urine, etc.

Depending upon the purpose of the sample, the sample node can be used to assist in purification of a sample applied thereto. For example, an elastomer from a sample node can be treated with a chemical stabilizer that lyses blood cells, thus releasing cellular DNA when blood is applied to the elastomer. Upon drying, the DNA forms a stable amorphous solid (with blood proteins) within the elastomer element. In the enhanced proteinaceous amorphous solid, DNA is stabilized at ambient temperature for at least 10 years, which is more than 100× longer than required to support the ambient-temperature transport of a sample carrier from collection site (e.g., by ordinary FEDEX or USPS) to a central storage or processing site. The stability of the DNA also allows adequate storage time to support nearly all biobanking applications.

At an appropriate processing site, the identifying indicia (e.g., bar code or radio-frequency tag) associated with sample carrier is re-read, to confirm the identity of the sample. The sample carrier is then unsealed, as necessary (e.g., a corresponding receptacle can be removed and set aside for recycling along with any drier pack located inside), and wash buffer is added to the elastomer, which remains positioned in the sample carrier. After soaking for 20 minutes at room temperature to rehydrate the sample, the elastomer is pressed to the bottom of the sample carrier with a blunt pipette tip, which compresses the elastomer sponge and releases a protein eluate into solution. The resulting solution is then drawn away by the pipette. Alternatively, the protein eluate can be separated from the elastomer by means of centrifugation (e.g., for a sample carrier having a cup-like morphology and an opening with a reservoir located beneath the sample node, direct centrifugation will result in the eluate being transferred to the reservoir for subsequent collection). Crucial to this process, high molecular weight DNA of interest is physically trapped within the pores of the elastomer. The addition of the wash solution, soaking, compression/centrifugation and withdrawal is repeated, thus producing a partially purified DNA product, still trapped in the elastomer pores. The final product, purified DNA, is released by addition of a high pH buffer and/or heat to the elastomer. The basic buffer and/or heating causes the pores of the elastomer to swell and release the purified DNA into solution. The DNA solution is then recovered by compression or centrifugation, as described above.

All of the processing steps just described could be performed with ordinary laboratory automation and, most importantly, the DNA thus obtained can be drawn from the subject, stabilized, shipped, stored and recovered in the same sample carrier. At no time would the specimen be refrigerated and, until the point at which the purified DNA is finally used for genetic analysis, the DNA would have resided in the same sample carrier throughout acquisition, shipping, storage, re-hydration and purification.

The ability to purify DNA within the same elastomer device that was used to ship and store the specimen is unique to the sample carriers of the present invention and is enabling in the context of very large scale sample acquisition. Increased processing rate and cost reduction is achieved due to the extreme simplification of workflow brought about by the elastomer-based purification technology and, in some instances, the elimination of add-on DNA purification technologies, such as magnetic beads or spin columns.

### Example 9

Human genetics is advancing at an exponential rate. The range of genetic knowledge, although already impressive, is predicted to double on a yearly basis over the next several decades. Thus, it is clear that we have entered a new era, where genetic principles and genetic testing to back them up, will become a routine part of daily life. The bioinformatics field is early enough in its development, that it is not clear what the full range of bioinformatics testing, such as genetic testing, will be, or, over time, what the spectrum of technologies will be to support the full range of testing that will emerge over the next twenty to fifty years.

At its heart, complex genetic testing is an example of sophisticated physical chemistry—the physical chemistry of the DNA polynucleotide strands—coupled to sophisticated informatics, which is used to assemble sequencing chemistry or to de-convolute hybridization binding interactions into gene sequence structure. In spite of its direct coupling to such “21^{st} century polymer chemistry,” the acquisition, transport, storage and purification of DNA is, for the most part, still treated like an exercise in functional biology. DNA-containing samples are treated as if they are “alive”, rather than polymer chains: they are shipped in the cold, stored in the cold, and subjected to purifying treatments that were developed in a 20^{th} century world of “wet” bench biology, rather than with an eye to supporting an extremely high-tech marriage of physical chemistry and computer science. As a result, high throughput, computer intensive, applied genetic analysis—the future of applied genetics—has become captive to, and ultimately bogged down by, the slow, expensive, arcane methods of 20^{th} century DNA sample collection, preservation, shipping, purification and release.

The technical vision that drives this invention is that DNA can be collected en masse from a population, such as a human population, a population of livestock, etc., by a painless finger prick or other standard method of obtaining a blood sample, to present a droplet of blood that is transferred by direct contact wicking into an engineered elastomeric (sponge) matrix, embedded in a storage system such as shown in FIG. 5. The sample carrier serves as a ergonomic device to present the elastomeric element to skin contact; the corresponding receptacle serves as a hardened vessel to protect the elastomer during transport, provides internal drying capacity to allow the specimen to solidify in situ; the storage system includes reference tracking signals embedded within the sample carrier and/or corresponding receptacle, and can hold the solidified blood specimen at room temperature in an archive system, if needed, for many years; and most importantly, the system is configured so that when the specimen, such as a DNA sample, is needed for analysis, the sample carrier has a structure that facilitates automated re-hydration, DNA purification and release. This integrated device concept, refer to as a “Chaperone Tube,” serves as an ergonomic device for large scale sample collection, storage and transport that is cost efficient, high throughput, computer driven applied bioinformatics analyses of biological samples.

The sample carriers and storage systems of the invention will find use in a wide variety of settings, including medical research, medical treatment, animal and plant breeding, veterinary medicine, food quality analysis, environmental screening, etc. One setting of interest is a military setting. From a military perspective, a remarkable range of human genetic diversity is becoming known, which could be used to identify, a priori, personal variation in endurance capacity, weight loss during training, muscle strength increase, wound healing rate, bone density, altitude sensitivity, risk of psychological disorder, response to infection, and response to medication. It is becoming clear that such knowledge will be put to work, very soon, to predict the strengths and weaknesses of the war fighter, while in service, and after retirement into civilian life.

Three general principles can be laid out, to guide the technical future of large-scale military testing, over the next 50 years. (1) Complexity: The genetic factors which lay beneath any important set of performance traits (endurance, speed, strength, wound healing, response to medication, infection risk, chemical sensitivity) will not be revealed by a simple single-gene test, but will involve analysis of relatively complex gene panels for each indication, probably at the allele level, rather than at the level of simple localized polymorphism; (2) Strategic Planning: Genetic testing will be correlated with well-defined, anticipated stress factors and military risks (hand warfare, altitude, cold, heat, risk of cutting, risk of burn, risk of exposure to a chemical or biological agent). Thus, high value genetic testing will not ordinarily be done in haste, on the battlefield, but will be done diligently, in preparation for combat, or at the time of recruitment, or during basic training; and (3) Centralized Testing: The technologies that will enable such military genetic testing will be very high throughput, and multiplex in nature, thus minimizing the amount of DNA that must be collected per individual. That kind of very high throughput, multiplexed analysis will almost certainly be performed at a few specialized sites, which, generally speaking, will be at great distance from any particular battlefield.

The present invention focuses on how general principles of solid state biospecimen management might be optimized to enable the rapid, low-cost, world-wide flow of DNA material as part of a secure, “hardened” military network. Thus, in a military recruiting/training camp, battlefield, or hospital, or during an emergency evacuation, The technical vision that drives this plan is that DNA can be collected from a soldier, or a recruit, or an ancillary civilian by a painless finger prick and then transferred by direct contact wicking into an engineered elastomeric (sponge) substrate held by a sealable and trackable sample carrier, part of the Chaperone Tube storage system described above.

To appreciate the value of Chaperone Tube technology, consider the following: A recruit, as part of the enlistment process, has agreed to be tested for what has evolved (by 2015) to be the 10 standardized panels of genetic performance markers. Each test panel comprises analysis of alleleic variation within each of 6 genes. The analysis is performed by fourth generation re-sequencing or microarray technology, which covers about 1 mB of the genome and will consume a total of about 1 μg of total DNA, which is readily obtained from a drop (50 μL) of human blood from a healthy volunteer. That testing is performed at a secure, regionalized, very high throughput genetic testing facility, which is more than 1000 miles away. The blood drop is presented by a painless finger prick, obtained while standing in line.

The Chaperone Tube, a molded plastic tube (i.e., receptacle) with a cap (i.e., sample carrier) which pulls off much like the cap of a USB jump drive, is opened to reveal a 6 mm (diameter)×5 mm (height) cylindrical elastomer element, positioned snugly and flush at the head of the opened tube cap. The elastomer is touched to the finger; the blood drop is transferred directly into the elastomer sponge by passive wicking; the tube is immediately re-capped; and the external reference tag (e.g., located on the sample carrier/cap) is read, to enter the specimen into the network. The body of the tube is pre-assembled with a drier pack within it, which upon closure, drives the evaporation of water from the encapsulated blood specimen, in several hours. The elastomer is treated with chemical stabilizers that lyse the blood cells, thus releasing the cellular DNA, which upon drying, forms a stable amorphous solid (with the blood proteins) within the elastomer element. In that enhanced proteinaceous amorphous solid, DNA is stabilized at ambient temperature for at least 10 years, which is more than 100× longer than required to support the ambient-temperature transport of the filled Chaperone Tube from the induction center (e.g., by ordinary FEDEX) to a central processing site.

At the processing site, the reference tag on the Chaperone Tube is re-read, to confirm identity, the tube is opened and discarded for recycling (along with the dryer pack inside it) and the desired DNA purified as described, e.g., in Example 8. In addition, as needed, protein and small molecule analytes can be collected from the proteinaceous wash solutions. Significantly, at no time would the specimen be refrigerated and, until the point at which the purified DNA is finally used for genetic analysis, the DNA would have resided in the same Chaperone Tube throughout acquisition, shipping, storage, re-hydration and purification.

### Example 10

Building the “Chaperone Tube” into a Biobank or Genetic Screening Network. The Chaperone Tube technology must account for the flow of genetic material from a very large number of collection sites, “Sources,” and routing to multiple specialized sites, “Receivers,” for storage or analysis. When described in this manner, it can be seen that national scale biobanking or societal-scale genetic testing is, in fact, an example of a network-based problem—one that is at least 1000 times more complex than our current understanding of the logistics of universal neonatal screening or HLA-typing. Accordingly, very large-scale biobanking and societal genetic testing must become formatted as a network, with properties similar to the flow of electronic information, as embodied in our current understanding of the Internet, and the way that the Internet is coupled to complex physical routing systems such as FEDEX or USPS.

For the purposes of discussion, we use the term “DNA-net”, to refer to the network solution that is required to collect, route, and distribute physical genetic information in the mid-to-late 21st century. A national or international scale system is needed to orchestrate the flow of physical genetic content, embodied as DNA strands in the solid state. With only minor technical and formatting modification, the same DNA-net would support many the many diverse applications shown in Table 4, as a secured network with access and interoperability that would be regulated by the same sort of password protection, encryption and firewalls that have been developed, very successfully, for the Internet.

TABLE 4
Genetics will become a central feature of U.S. society by 2015

| Application Area | 2009 Status | 2015 Status (projected) |
|---|---|---|
| Medical Treatment- | solid organ & marrow | solid organ & marrow, 100K tests/yr |
| Transplantation | | |
| Medical Treatment- | R&D only | Neuro-degenerative, CV, plastic |
| Stem Cells | | surgery: genetics to determine a |
| | | match, 100K tests/yr |
| Medical Treatment- | warfarin, abacavir | Nearly all drugs based on liver |
| Pharmaceutics | | clearance, immunological rash, |
| | | receptors, 1 MM tests/yr |
| Medical Treatment- | R&D only | Universal HLA screening for |
| Vaccination | | childhood and adult vaccine response, |
| | | 10 MM tests/yr |
| Medical Treatment - | BRACA, EGFR (cancer) | All cancers, all CV indications, |
| Diagnostics | | rheumatology, 1 MM tests/yr |
| Neonatal Screening | CF, cycle cell, Tay Sachs | HLA and at least 5 others, universally |
| | | at birth. This will be the 21^{st} century |
| | | analogue of the ABO blood type, |
| | | 1 MM tests/yr |
| Public Health- | MRSA, classical petrie | HIV, flu, dengue, West Nile |
| Infection Risk | dishes analytical culture for | sensitivity, MRSA, drug resistant TB |
| | the rest | risk, 1 MM tests/yr |
| Public Health-Cancer | R&D | Liver genes (carcinogen clearance) |
| Risk | | Immune markers, DNA repair, 1 MM |
| | | tests/yr |
| Public Health- | R&D | Ghrelin, adipocyte stem cells, |
| Obesity Risk | | metabolism, neuro-addictive |
| | | screening, 1 MM tests/yr |
| Environmental | Classical petrie dishes & | Genetic testing of 40,000 sources per |
| Testing-Water | culture | day in U.S. will replace cell culture, |
| | | 10 MM tests/yr |
| Environmental | Classical petrie dishes & | Flu, drug resistant TB, will replace |
| Testing-Air | culture | cell culture, 1 MM tests/yr |
| Environmental | Classical petrie dishes & | Microbial biodiversity via |
| Testing-Soil | culture | microarrays, |
| | | to monitor contamination, 100K |
| | | tests/yr |
| Climate Change- | Smthsonian & NSF bar | Worldwide genetic biodiversity |
| Biodiversity | code of life project | testing, especially among sentinel |
| | | microbes, 1 MM tests/yr |
| Food Testing-food | Some PCR but classical | Microbial screening for all domestic |
| borne disease | petrie dishes & culture | and imported food stock: factories & |
| | dominate | point of entry via PCR, beads, |
| | | microarrays, 50 MM tests/yr |
| Food Testing-genetic | Genetically engineered | Large scale screening of |
| modification | plants: QC and industrial | environmental back crossing to wild |
| | piracy | stock, 10 MM tests/yr |
| Forensics-casework | All ID via Identifiler | ID via Identifiler & trait & clan |
| | | analysis, 10 MM tests/yr |
| Forensics-ID | All violent offenders | All booked offenders, 1 MM tests/yr |
| databases | | |
| Defense-Military | Identification of the dead | Identification of the dead, |
| | | performance trait screening of all |
| | | recruits, 100K tests/yr. |
| Defense-Bioterrorism | R&D | Bioshield air and water screening for |
| | | militarized pathogens, 1 MM tests/yr |
| Defense-Immigration | R&D | Current French model: DNA ID on all |
| | | visa applicants, 1 MM tests/yr |
| R&D-Human | Traditional lab R&D on | National discovery biobanking similar |
| Genetics | small | to UK, Spain, France, Canada, Lux, |
| | ad hoc biobanks | Singapore, Japan, Malasia, Australia, |
| | | 1 MM tests/yr |
| R&D-Animal | Chicken & bovine | Expansion to all feedstock for marker |
| Breeding | quantitative trait selection | based selection, 10 MM tests/yr |
| | at DNA level | |
| R&D-Plant Breeding | Corn feedstock | Corn, soybean, wheat, rice and fuel |
| | | stock for food and bio fuels, 10 MM |
| | | tests/yr |

What does the DNA-net look like as a complex structure? The DNA-net has an interesting formal analogy to other complex networks that had been developed to move information, both LAN and the Internet, and would control the complex flow of genetic material, much as the internet-enabled FEDEX or USPS models control the complex flow of large physical packages. Below, the standard descriptive formalism of the Internet is used to describe the 3 network components that are needed to establish a DNA-net.

A) Transduction of Content into a Standard. In the Internet, the underlying format is based on the transduction of diverse information types (the content) into a standardized binary code. In the DNA-net, the physical formatting standard is the transduction of DNA in diverse sample types (the content) into a standardized solid-state format. The elastomeric sponge, held by sample carriers of the invention (e.g., such as shown in FIGS. 5 and 7), can be the content format for the DNA-net.

B) Formatting of Content for Transport & Tracking. In the Internet, all binary code content is formatted into a standardized “Packet”, where the binary content is parsed into a standard size (the “Payload”) and wrapped with a “Header”, which includes important information about the Payload. The Header has a standard information format which includes a description of the type of content that is in the Payload, where it has come from, and where it must go. In the DNA-net, solid-state DNA content is parsed into a Payload format and size (e.g., a solid DNA aliquot in an sample node having a standard composition and one of several standard dimensions) and the Header function is embodied in a tag (e.g., a radio-frequency tag) attached to a sample carrier which contains the sample node. As with the Header of a standard TCP/IP Internet Packet, the tag identifies the type of content in the Chaperone Tube (DNA from blood, DNA from saliva, DNA from plants, DNA from water filtrate, etc), where the content came from (a hospital, a police station, a water treatment plant); and where the content must go (a centralized medical testing facility, a crime lab, a water analysis lab). The ability to format data as a Packet is the underlying core technology of the Internet. Similarly, the storage system/Chaperone Tube, as defined herein, can be the “Packet” (i.e., the enabling core technology) of the DNA-net.

C) The Router. In the Internet, Packets are shipped throughout a network linked by nodes, where each node operates as a Router that accepts Packets that have been delivered to the node and then routes them to the destination address specified on the Header, by the fastest route possible. In the Internet, there are two functionally distinct types of Router: a) Local Routers that manage the flow of Packets within a local network, and b) Network Routers that manage the flow of Packets between local networks. Generally, the Local and Network Routers are owned by different institutions. Companies, universities, or government labs may own the Local Router, which operates on a cable network system also owned by the institution. The Network Router, in contrast, may be owned by an Internet Service Provider (ISP), such as Earthlink, which uses a physical network that is based on fiber optics or satellites. The physical network of the Network Router may be owned by a third party, such as a phone company. In the DNA-net, the Local Router is a new type of standardized automated system, developed to manage, store and retrieve Chaperone Tubes on demand, while the Network Router is provided by FEDEX or USPS, exactly as we know them. The Network Routers in the DNA-net route Chaperone Tubes from a Source to a designated Receiver, via the existing physical network system (highways, air-routes) that are owned by third party, usually the Federal Government.

The disclosures of all US patents and applications specifically identified herein are expressly incorporated herein by reference. To the extent that any definitions in the incorporated references are inconsistent with the definitions provided herein, the definitions provided herein are controlling. Particular features of the invention are emphasized in the claims which follow.

What is claimed:

1. A sample carrier comprising an opening and a sample node, wherein the opening is configured to hold the sample node while providing a ventilation space to a surface of the sample node, and wherein the sample node is held by the opening.

2. The sample carrier of claim 1, wherein the opening has a side surface that contacts the sample node, and wherein the sample node is held by such contact.

3. The sample carrier of claim 2, wherein the opening is configured to minimize the surface area of the sample node contacted by the opening.

4. The sample carrier of claim 1, wherein the opening comprises an open circle or open polyhedral in cross-section.

5. The sample carrier of claim 1, wherein the opening comprises 1, 2, 3, 4, 5, 6, or more sub-opening spaces.

6. The sample carrier of claim 3, wherein said sub-opening spaces facilitate fluid evaporation and/or air flow at the surface of the sample node.

7. The sample carrier of claim 1, wherein the opening comprises 1, 2, 3, 4, 5, 6, or more protrusions.

8. The sample carrier of claim 1, wherein the sample node comprises a macroporous medium.

9. The sample carrier of claim 8, wherein the macroporous medium has porosity in the 10 to 100 micrometer range.

10. The sample carrier of claim 8, wherein the macroporous medium is an elastomeric substrate.

11. The sample carrier of claim 8, wherein the macroporous medium is a cellulose-based filter paper.

12. The sample carrier of claim 1, wherein the sample node comprises a stabilizer.

13. The sample carrier of claim 12, wherein the stabilizer comprises a filler, a reactive oxygen scavenger, a detergent, an emulsifier, a chelator, a buffer, or any combination thereof.

14. The sample carrier of claim 12, wherein the filler is sucrose or trehalose, the reactive oxygen scavenger is histidine or pyruvate, the detergent is a strong anionic detergent or a weak non-ionic detergent, and the buffer had a pH of about 6.5 to about 8.5 or about 4.0 to about 6.0.

15. The sample carrier of claim 1, wherein the sample node comprises an identifying indicia.

16. The sample carrier of claim 1, wherein the sample node comprises a sample node cavity to increase its surface area.

17. The sample carrier of claim 1, wherein at least 70% of the surface of the sample node is exposed to air.

18. The sample carrier of claim 1, wherein the sample node has a fluid holding capacity of about 150 to about 1500 microliters.

19. The sample carrier of claim 1, wherein the sample carrier comprises an identifying indicia.

20. The sample carrier of claim 1, further comprising a biological sample carried by the sample node.

21. The sample carrier of claim 20, wherein the biological sample is from a human, a lab animal, a farm animal, a zoo animal, or a wild animal.

22. The sample carrier of claim 20, wherein the biological sample is a blood sample, a serum sample, a plasma sample, a buccal sample, a sputum sample, a nasal swab, a milk sample, a homogenized plant or animal tissue, or a cell lysate.

23. The sample carrier of claim 1 comprising a plurality of openings and sample nodes.

24. A sample carrier comprising an opening configured to hold a sample node while providing a ventilation space to a surface of the sample node.

25. The sample carrier of claim 24, wherein the opening comprises at least 1, 2, 3, 4, 5, 6 or more sub-opening spaces.

26. The sample carrier of claim 24, wherein the opening comprises at least 1, 2, 3, 4, 5, 6 or more protrusions.

27. A storage system comprising:
a sample carrier of claim 1; and
a receptacle,
wherein the sample carrier can interface with the receptacle.

28. The storage system of claim 27, wherein the interface between the sample carrier and the receptacle forms a sealed enclosure around the sample node.

29. The storage system of claim 28, wherein the receptacle comprises a desiccant.

30. The storage system of claim 27, wherein the sample carrier fits into the receptacle.

31. A method of collecting a biological sample, comprising applying a biological sample to a sample node of a sample carrier of claim 1.

32. The method of claim 31, further comprising allowing the biological sample to dry upon the sample node.

33. The method of claim 31, wherein the biological sample is a blood sample, a serum sample, a plasma sample, a buccal sample, a sputum sample, a nasal swab, a milk sample, a homogenized plant or animal tissue, or a cell lysate.

34. The method of claim 31, comprising sealing the sample carrier such that the sample node is isolated from external sources of contamination.

35. The method of claim 34, further comprising sterilizing the sealed sample carrier.

36. A method of recovering a biological sample, comprising ejecting a sample node out of a sample carrier of claim 1 by pushing it or pulling it out of the opening.

37. The method of claim 36, further comprising adding water to the sample node to re-hydrate the sample.

38. The method of claim 36, further comprising shipping the biological sample from a first location where it is collected to a second location where it is recovered.

39. A kit comprising a sample carrier of claim 1.

40. A kit comprising a storage system of claim 27.
